(19) United States
(12) Patent Application Publication
(10) Pub. No.: US 2023/0049215 A1
(43) Pub. Date: Feb. 16, 2023

(54) CATALYTIC NUCLEIC ACID-BASED GENETIC ENGINEERING METHOD

(71) Applicant: The Board of Trustees of the University of Illinois, Urbana, IL (US)

(72) Inventors: Yi Lu, Austin, TX (US); Mingkuan Lyu, Champaign, IL (US)

(73) Assignee: The Board of Trustees of the University of Illinois, Urbana, IL (US)

(21) Appl. No.: 17/786,865

(22) PCT Filed: Dec. 18, 2020
(86) PCT No.: PCT/US2020/066223

§ 371 Date: Jun. 17, 2022

Related U.S. Application Data
(60) Provisional application No. 62/950,863, filed on Dec. 19, 2019.

(51) Int. Cl. C12N 15/10; C12N 15/113

(52) U.S. Cl. CPC C12N 15/102; C12N 15/113; C12N 2310/12

(57) ABSTRACT

Systems and kits are disclosed herein for genetic engineering (such as for DNA cleavage and gene-editing), which include catalytic nucleic acids and catalytic nucleic acid-assisting reagents. Methods of genetic engineering are also described, in which both catalytic nucleic acid-assisting reagents and catalytic nucleic acids are specific for a target site, thus, providing high-fidelity genetic engineering.

### CROSS REFERENCE TO RELATED APPLICATIONS

This application claims the benefit of U.S. Provisional Patent Application No. 62/950,863, filed Dec. 19, 2019, which is incorporated herein by reference in its entirety.

### ACKNOWLEDGMENT OF GOVERNMENT SUPPORT

This invention was made with government support under DE-SC0018420 awarded by The Department of Energy. The government has certain rights in the invention.

### FIELD

This application provides systems, kits, and methods, which include catalytic nucleic acids, for genetic engineering of nucleic acids.

### BACKGROUND

Genetic engineering methods often involve sequence-specific manipulation of nucleic acids by protein enzymes. Commonly used protein-based restriction enzymes lack customizability, which can be solved by programmable protein-based endonuclease systems, such as TALEN or CRISPR/Cas. However, these protein-based systems have limitations, including engineering difficulties (e.g., TALEN) and off-target effects (e.g., CRISPR/Cas). In addition, since protein enzymes such as Cas are relatively large, they may not be able to access targets located in areas with limited space, such as genes tightly packed in chromatin. Catalytic nucleic acid-based systems have emerged as a viable alternative. For example, DNAzymes have proven to be smaller, more stable and cost-effective than protein-based restriction enzymes. As a result, they have been used for many applications, such as biosensing, environmental monitoring, medical diagnostics, and bioimaging in cells and animals. Despite the progress made, most of these applications are limited to cleaving single-stranded DNA (ssDNA) or RNA, and the activity of catalytic nucleic acids on double-stranded DNA (dsDNA) has never been verified. This has hindered their application in direct gene manipulation, such as gene cloning and editing, where the major form of the targets is dsDNA.

### SUMMARY

In order to advance genetic engineering, DNA manipulation technologies with higher fidelity, customizability and gene accessibility are desirable. Therefore, disclosed herein are systems and methods for genetic engineering. The systems disclosed herein include one or more catalytic nucleic acids (e.g., one or more DNAzymes or ribozymes) and one or more catalytic nucleic acid-assisting reagents. In some embodiments, the systems include PNA-Assisted Double-Stranded DNA Nicking by DNAzymes (PANDA) or ssDNA-Assisted double-stranded DNA Nicking by DNAzymes (DANDA). In embodiments, the one or more catalytic nucleic acids include one or more catalytic domains and one or more nucleic acid binding domains. In examples, the one or more binding domains include a sequence complementary to and/or identical to a sequence flanking a target modification site and/or within the target site. In examples, the one or more catalytic nucleic acid-assisting reagents include a sequence that specifically binds a sequence that is complementary to and/or identical to a sequence flanking the target site and/or within the target site.

In certain embodiments, catalytic nucleic acids include DNAzymes. In embodiments, one or more catalytic nucleic acids can include one or more DNAzymes (such as at least one or at least two DNAzymes). In examples, the catalytic domain of the DNAzyme includes DNA hydrolysis activity. In embodiments, a first binding domain that specifically binds a sequence flanking the target site (e.g., on the 5′ side of the target site), and a second binding domain that specifically binds a sequence flanking the target site on the opposite side compared with the first binding domain (e.g., on the 3′ side of the target site). Additional catalytic domains are possible, such as catalytic domains with phosphodiester hydrolytic cleavage, oxidative cleavage, nucleoside excision, phosphorylation, or ligation activity. In specific, non-limiting examples, the one or more DNAzymes include a 13PD1 catalytic domain sequence, such as a sequence including residues 10-50 of SEQ ID NO: 5. In other non-limiting examples, the one or more DNAzymes include a 13PB2 catalytic domain sequence, such as a nucleic acid sequence including residues 10-50 of SEQ ID NO: 37. In other non-limiting examples, the one or more DNAzymes include a I-R3 catalytic domain sequence, such as a nucleic acid sequence including residues 10-19 of SEQ ID NO: 54.

In some embodiments, the one or more catalytic nucleic acid-assisting reagents include peptide nucleic acids (PNAs), such as bis-PNAs, pseudo-complementary PNAs (pc-PNAs), tail-clamp PNAs (tc-PNAs), and/or 7-PNAs, or analogs thereof, such as polyacrylate nucleic acid analogs and/or nucleobase-containing polymers with polyester, polyvinyl, or polyamide backbones. In other embodiments, the catalytic nucleic acid-assisting reagents include one or more single-stranded assisting DNAs (saDNAs). In other embodiments, the one or more catalytic nucleic acid-assisting reagents include small molecules or oligomers that specifically bind dsDNA including dsDNA minor-groove-binding polyamides. In examples, dsDNA minor-groove-binding polyamides contain N-methylimidazole and N-methylpyrrole amino acids. In some embodiments, dsDNA minor-groove-binding polyamides include eight-ring pyrrole-imidazole polyamides. In other embodiments, the one or more catalytic nucleic acid-assisting reagents include nucleic acids, such as DNA, RNA, or analogs thereof, with modified terminals or nucleotides. In examples, the modified nucleotides include 2′-O-(Pyren-1-yl)methyl-RNA monomers.

In embodiments, the one or more catalytic nucleic acid-assisting reagents can form Watson-Crick and/or Hoogsteen base pairs with a sequence that is complementary to and/or identical to a sequence flanking the target site and/or within the target site. In some examples, the one or more catalytic nucleic acid-assisting reagent sequences can form a triple helix with a sequence complementary to the sequence flanking a target site and/or the target site sequence. In other examples, the one or more catalytic nucleic acid-assisting reagent sequences can form a double helix with a sequence complementary to the sequence flanking a target site and/or the target site sequence.

Kits are further disclosed herein. In some examples, the kits include one or more catalytic nucleic acids and one or more catalytic nucleic acid-assisting reagents of the systems disclosed herein. In specific, non-limiting examples, the kits include one or more DNAzymes and one or more PNAs of the systems disclosed herein. In other non-limiting examples, the kits include one or more DNAzymes and one or more saDNAs of the systems disclosed herein.

Methods of genetic engineering are also disclosed. In some examples, the methods include contacting a target nucleic acid with any of the systems disclosed herein under conditions sufficient for specific binding of the one or more catalytic nucleic acid-assisting reagents (such as one or more PNAs or one or more saDNAs) to the target nucleic acid and under conditions sufficient for catalytic nucleic acid activity (such as DNAzyme activity).

Additional methods of genetic engineering are disclosed. In embodiments, the methods include contacting a target nucleic acid (such as double-strand DNA, for example, genomic DNA) with one or more catalytic nucleic acid-assisting reagents (such as PNAs). In examples, the one or more catalytic nucleic acid-assisting reagents include a sequence that specifically binds a sequence that is complementary to and/or identical to a sequence flanking a target site and/or within a target site. In examples, the contacting can occur under conditions sufficient for specific binding between the target nucleic acid and the one or more catalytic nucleic acid-assisting reagents, thus, generating a catalytic nucleic acid-assisting reagent-bound target nucleic acid. The methods further include contacting the catalytic nucleic acid-assisting reagent-bound target nucleic acid with one or more catalytic nucleic acids. For example, the one or more catalytic nucleic acids can include one or more catalytic domains and one or more binding domains, in which the one or more binding domains include a sequence complementary to and/or identical to a sequence flanking the target site. In examples, the contacting can occur under conditions sufficient for catalytic nucleic acid activity.

In embodiments, reaction conditions for the ssDNA-Assisted double-stranded DNA Nicking by DNAzymes (DANDA) system include a pH of 6-8, high salts, and 40° C.-95° C., ensuring specific binding between catalytic nucleic acid-assisting reagents (e.g., saDNAs) and the target nucleic acids. In other embodiments, reaction conditions sufficient for optimized activity of DANDA include 630 mM Na⁺, 2.5 mM Mg²⁺, pH 7.5, and 75° C. In further examples, the target nucleic acid can be contacted with the one or more saDNAs for at least 5 minutes.

In embodiments, reaction conditions for the PNA-Assisted Double-Stranded DNA Nicking by DNAzymes (PANDA) system include a pH of 6-8, low or no salts, and 4° C.-40° C., ensuring specific binding between catalytic nucleic acid-assisting reagents (e.g., bis-PNA and/or γ-PNA) and the target nucleic acid. In one example, conditions sufficient for specific binding between bis-PNA or γ-PNA is pH 6.8-7.5, 0.5-2 mM Zn²⁺ at 20-37° C., for use with the DNAzyme I-R3. In some non-limiting examples, the conditions sufficient for DNAzyme activity can include a pH of 7.5, 1 mM ZnCl₂, 20 mM MnCl₂, and 25° C. In further examples, the target nucleic acid can be contacted with the one or more PNAs for at least 20 minutes.

The methods disclosed herein can be performed in a variety of environments, for example, the methods can be performed in vitro, ex vivo, in vivo, or in cellulo. In some embodiments, the target nucleic acid is genomic DNA present in a cell.

Further still, methods of treating a disease or disorder are disclosed herein (such as methods treating a subject with a disease or disorder). For example, the methods can include administering the system to a subject, thereby treating the disease or disorder.

### BRIEF DESCRIPTION OF THE DRAWINGS

FIGS. 1A-1C show (FIG. 1A) a programmable DNA endonuclease using the CRISPR/Cas9 system, (FIG. 1B) a programmable DNA nicking system using an exemplary embodiment of the disclosed system (‘the PANDA system’), and (FIG. 1C) a recognition pattern of one example of the PANDA system (PANDA-A), further showing: 13PD1-1: 5′AGAGGAAGCTATACCGGGCAACTATTGCCTCGTCATCGCTATTTTCTGCGAGGAGGA GA (SEQ ID NO: 5); PNA1: H-KK-TCTCCTCC-(eg₁)₃-JJTJJTJT-K—NH₂ (SEQ ID NO: 1); PNA2: H-KK-TJTJJTTJ-(eg₁)₃-CTTCCTCT-K—NH₂ (SEQ ID NO: 2); PANDA-A Target Site Sense Strand 1: 5′TCTCCTCCTACTGCTGCTTCCTCT (SEQ ID NO: 28); PANDA-A Target Site Antisense Strand 1: 3′AGAGGAGGATGACGACGAAGGAGA (SEQ ID NO: 29).

FIGS. 2A-2C show a plasmid nicked by an exemplary PANDA-A system with the recognition sites on plasmid 1 (FIG. 2A), an agarose gel analysis of the plasmid nicked by PANDA-A (FIG. 2B), and (FIG. 2C) an agarose gel analysis of plasmids with recognition sites mismatched with PANDA-A and nicked by PANDA-A, further showing alignments of SEQ ID NOS: 28 (top line) and 29 (bottom line) (full match), SEQ ID NOS: 28-29 with a mismatch at nucleotide 5 (PNA1 region mismatch), SEQ ID NOS: 28-29 with a mismatch at nucleotide 20 (PNA2 region mismatch), and SEQ ID NOS: 28-29 with mismatches at nucleotides 5 and 20 (PNA1+PNA2 mismatches).

FIGS. 3A-3B show a plasmid nicked or linearized by two exemplary PANDA systems, PANDA-A and PANDA-B, with (FIG. 3A) recognition sites on plasmid 2 and (FIG. 3B) an agarose gel analysis of plasmid 2 nicked by PANDA-A or PANDA-B or linearized with both PANDA-A and PANDA-B, further showing SEQ ID NOS: 28-29 (PANDA-A) as well as PANDA-B, 5-′TTCTCTTCTACTGCTGCCTTTCTT-3′ (SEQ ID NO: 30) and 3′-AAGAGAAGATGACGACGGAAAGAA-5′ (SEQ ID NO: 31).

FIGS. 4A-4C show an example application of PANDA-based genetic engineering in molecular cloning, in which PANDA systems can mimic restriction enzymes (FIG. 4A). FIG. 4B shows an agarose gel analysis of plasmid 3 fragmentized with PANDA systems. FIG. 4C shows a sequencing analysis of recombinant plasmid 3 which demonstrate successful recombination, further showing the following sequences:

A fragment of plasmid 3:
(SEQ ID NO: 32)
5′-GATCATTTCTCTTCTACTGCTGCCTTTCTTATAGAGGAAGCAGCAGT
AGGAG..................AAGAAAGGCAGCAGTAGAAGAGAAATT
CTCCTCCTACTGCTGCTTCCTCTAAGCT-3′
(SEQ ID NO: 33)
3′-CTAGTAAAGAGAAGATGACGACGGAAAGAATATCTCCTTCGTCGTCA
TCCTC..................TTCTTTCCGTCGTCATCTTCTCTTTAA
GAGGAGGATGACGACGAAGGAGATTCGA-5′
Recombinant plasmid 3:
(SEQ ID NO: 34)
5′-GATCATTTCTCTTCTACTGCTGCCTTTCTTATAGAGGAAGCATATAC
CTCGCATTGGAGCTAGCTACATGTTGGCACTGGGCAGTAGAAGAGAAATT
CTCCTCCTACTGCTGCTTCCTCTAAGCT-3′
(SEQ ID NO: 35)
3′-CTAGTAAAGAGAAGATGACGACGGAAAGAATATCTCCTTCGTATATG
GAGCGTAACCTCGATCGATGTACAACCGTGACCCGTCATCTTCTCTTTAA
GAGGAGGATGACGACGAAGGAGATTCGA-5′

FIG. 5 shows a γ-PNA PANDA system, including 13PB2-1, γ-PNA1 and a dsDNA target sequence. γ-PNA benefits from enhanced DNA binding properties. The target site sequence requirements are less stringent for γ-PNA and 13PB2-1.

FIGS. 6A-6B show a plasmid containing four γ-PNA PANDA sites in two pairs (FIG. 6A) treated by corresponding γ-PNA PANDA systems. A γ-PNA PANDA system including γ-PNA1, γ-PNA2, 13PB2-1, and 13PB2-2 (FIG. 6A) generates a DSB break in pEGFP plasmid, resulting in a linear band (lane 4) (FIG. 6B).

FIGS. 7A-7B shows a programmable DNA nicking system using another exemplary embodiment of the disclosed system (‘the DANDA system’) enabling two DNAzymes to cleave opposing strands of dsDNA target via a double-nicking reaction (FIG. 7A). FIG. 7B shows that only pEGFP plasmid incubation with saDNA1, saDNA2, 13PB2-1, and 13PB2-2 generates ˜3 kb and ˜2 kb dsDNA fragment after treatment of BsaI, indicating a DSB break made by the DANDA system before BsaI treatment.

FIG. 8 depicts, in a DANDA system, the effect of saDNA length on nicked product formation. The gel analysis shows that increasing saDNA length biases the equilibrium toward cleaved product. FIG. 8 shows saDNAs of increasing length including: saDNA3 (SEQ ID NO: 44) (lane 1 & 2), saDNA4 (SEQ ID NO: 45) (lane 3 & 4), saDNA5 (SEQ ID NO:46) (lanes 5 & 6), and saDNA6 (SEQ ID NO: 47) (lane 7 & 8).

24 nt saDNA3:
(SEQ ID NO: 44)
5′-TCTCCTCCTACTGCTGCTTCCTCT-3′
50 nt saDNA4:
(SEQ ID NO: 45)
5′-CGCCTCAGCCGTCTCCTCCTACTGCTGCTTCCTCTCGGAATGCTACT
TCG-3*
70 nt saDNA5:
(SEQ ID NO:46)
5′ CTAACGGATTCGCCTCAGCCGTCTCCTCCTACTGCTGCTTCCTCTCG
GAATGCTACTTCGTGTATGCGGC-3*
90 nt saDNA6:
(SEQ ID NO: 47)
5′-CGGCACCTCGCTAACGGATTCGCCTCAGCCGTCTCCTCCTACTGCTG
CTTCCTCTCGGAATGCTACTTCGTGTATGCGGCGACCGAGTTG-3′

FIGS. 9A-9C show an example of PANDA-based recombineering in living bacteria. Specifically, pReporter (FIG. 9A), which includes a kanamycin resistance gene with a premature stop codon, and a repair template are introduced into E. coli HME 63 including Exo, Gamma, and Beta genes (FIG. 9B). When the PANDA system includes PNA, DNAzyme, and a template strand (lane 4), the cells recover Kanamycin resistance by homologous recombination with the template (FIG. 9C).

### SEQUENCE LISTING

Any nucleic acid and amino acid sequences listed herein or in the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NOs: 1-4 are exemplary catalytic nucleic acid-assisting reagents.

SEQ ID NOs: 5, 6, 8 are exemplary catalytic nucleic acids with flanking targeting sequences.

SEQ ID NOs: 7, 9, 56, and 57 are a DNA oligonucleotide negative controls.

SEQ ID NOs: 10-13 are exemplary primers.

SEQ ID NOs: 14-19 are exemplary templates.

SEQ ID NOs: 20 and 21 are molecular cloning insert fragments with cohesive ends (underlined) and NheI recognition sites (underlined italic).

SEQ ID NOs: 22-27 are representative sequences of exemplary plasmids.

SEQ ID NOs: 28 and 29 is a PANDA-A target site, including sense and antisense DNA strands.

SEQ ID NOs: 30 and 31 is a PANDA-B target site, including sense and antisense DNA strands.

SEQ ID NOs: 32 and 33 are fragments of plasmid 3.

SEQ ID NOs: 34 and 35 are representative sequences of recombinant plasmid 3.

SEQ ID NOs: 36 and 59 are γ-PNA1 and 7-PNA2, respectively.

SEQ ID NOs: 37 and 60 are 13PB2-1 and 13PB2-2, respectively

SEQ ID NO: 38 is an example of target sequence which matches both PNA1 and PNA2.

SEQ ID NO: 39 is an example of target sequence which has a mismatched base with PNA1.

SEQ ID NO: 40 is an example of target sequence which has a mismatched base with PNA2.

SEQ ID NO: 41 is an example of target sequence which has a mismatched base with PNA1 and a mismatched base with PNA2.

SEQ ID NO: 42 is an exemplary saDNA strand (“saDNA1”).

SEQ ID NO: 43 is an exemplary saDNA strand (“saDNA2”).

SEQ ID NO: 44 is an exemplary saDNA strand (“saDNA3”).

SEQ ID NO: 45 is an exemplary saDNA strand (“saDNA4”).

SEQ ID NO: 46 is an exemplary saDNA strand (“saDNA5”).

SEQ ID NO: 47 is an exemplary saDNA strand (“saDNA6”).

SEQ ID NOs: 48-53 are exemplary primers used for PANDA-based recombineering.

SEQ ID NOs: 54 and 55 are exemplary I-R3 DNAzymes used for PANDA-based recombineering.

SEQ ID NO: 58 is a control oligomer used for PANDA-based recombineering.

SEQ ID NO: 61 is a homologous recombination template used for PANDA-based recombineering.

### DETAILED DESCRIPTION

The present disclosure provides methods of site-specifically nicking and cleaving dsDNA, using DNAzymes and PNA in a system named PNA-Assisted Double-Stranded DNA Nicking by DNAzymes (PANDA) or using DNAzymes and saDNA in a system named ssDNA-Assisted Double-Stranded DNA Nicking by DNAzymes (DANDA). Embodiments in PANDA include bis-PNA “openers” which have been shown to invade dsDNA and expose single-stranded regions by forming triplexes with the complement. In some examples, a DNA-hydrolyzing DNAzyme is used bearing the catalytic core of 13PD1, which offers rapid hydrolysis of specific phosphodiester bonds on ssDNA. Said PANDA systems also include variants where γ-PNAs are involved, in addition to a variety of DNAzyme catalytic domains including 13PD1, 13PB2, and I-R3. Embodiments in DANDA include variants of PANDA where PNAs are replaced by saDNAs.

These PANDA or DANDA systems are efficient in both nicking dsDNA and causing double stranded DNA breaks. The resulting dsDNA fragment can be ligated with another fragment for DNA recombination. Importantly, said PANDA systems have less off-target cleavage activity compared with CRISPR/Cas9 nickase (CRISPR/Cas9n) in nicking DNA and promote recombineering-based gene editing (e.g., in E. coli), making it a potentially more precise genetic engineering system. Finally, modified nucleotides, varying strand length, ionic strength and other techniques such as in vitro selection can be used to optimize both the catalytic nucleic acid and catalytic nucleic acid-assisting reagent, providing PANDA and DANDA systems with tunable catalytic activity.

### I. Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a DNAzyme” includes single or plural DNAzymes and is considered equivalent to the phrase “comprising at least one DNAzyme.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including journal articles, patents, and patent publications, and GenBank® Accession numbers cited herein are incorporated by reference in their entirety.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided.

Administer: As used herein, administering a composition to a subject means to give, apply, or bring the composition into contact with the subject. Administration can be accomplished by any of a number of routes, such as, for example, topical, oral, subcutaneous, intramuscular, intraperitoneal, intravenous, intrathecal, and intradermal.

Complementary: Ability to form base pairs between nucleic acids. Oligonucleotides and their analogs hybridize by hydrogen bonding, which includes Watson-Crick, Hoogsteen or reversed Hoogsteen hydrogen bonding, between complementary bases. Generally, nucleic acid molecules consist of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. Artificially or naturally modified nitrogenous bases can be involved. For example, pseudoisocytosine (J), or 5-methylcytosine (5mC) will hydrogen bond to G. “Complementary” refers to the base pairing that occurs between two distinct nucleic acids or two distinct regions of the same nucleic acid. In some examples, “complementary” also refers to triple-stranded nucleic acid (or triplex nucleic acid).

“Specifically hybridizable” and “specifically complementary” are terms that indicate a sufficient degree of complementarity such that stable and specific binding occurs between a nucleic acid (or its analog) and another nucleic acid target (e.g., DNA or RNA). The nucleic acid or analog may, but need not have, 100% complementarity to its target sequence to be specifically hybridizable. A nucleic acid or analog is specifically hybridizable when there is a sufficient degree of complementarity to avoid non-specific binding of the nucleic acid or analog to non-target sequences under conditions where specific binding is desired, for example in the methods disclosed herein. Such binding is referred to as specific hybridization.

Contact: Placement in direct physical association; includes both in solid and liquid form.

Catalytic nucleic acids: Catalytic nucleic acids are nucleic acid molecules that are capable of catalyzing a specific chemical reaction (such as oxidative cleavage or hydrolytic cleavage, for example, phosphodiester hydrolytic cleavage, nucleoside excision, phosphorylation (or de-phosphorylation), ligation, or other reactions). Catalytic nucleic acids include ribozymes (catalytic RNA or RNAzymes), DNAzymes (catalytic DNA or deoxyribozymes), and other natural or unnatural, modified, or unmodified nucleic acid molecules that are catalysts such as ZNAzymes, for example, arabino nucleic acid (ANA)-zymes, 2′-fluoroarabino nucleic acid (FANA)-zymes, hexitol nucleic acid (HNA)-zymes, and cyclohexene nucleic acid (CeNA)-zymes. In some examples, the catalytic nucleic acids (such as DNAzymes and/or ribozymes) can be allosteric (see, e.g., Soukup G. A., Allosteric Ribozymes as Molecular Switches and Sensors. In: Nucleic Acid Switches and Sensors, Springer, Boston, Mass., 2006, incorporated herein by reference in its entirety). Catalytic nucleic acids mimic proteins as enzymes, but the catalytic activity is due to a specific arrangement of nitrogenous bases, rather than amino acid residues, such as in proteins.

Catalytic nucleic acids herein can include additional elements, such as elements that guide the catalytic to a target site, for example, binding domains that specifically bind sequences identical to and/or complementary to a sequence within or flanking a target site. Catalytic nucleic acids can be used under a variety of conditions, such as conditions sufficient for catalytic activity (for example, DNAzyme activity), for example, under in vitro, ex vivo, in vivo, or in cellulo conditions.

Catalytic nucleic acid-assisting reagent: A reagent that assists catalysis or contact between a catalytic nucleic acid and a target. Catalytic nucleic acid-assisting reagents can include binding domains that bind a sequence that is complementary to or identical to a sequence within and/or flanking a target site. Catalytic nucleic acid-assisting reagents can include natural nucleic acids, such as DNAs and RNAs, or unnatural (modified) nucleic acids, such as peptide nucleic acids (PNAs), locked nucleic acids (LNAs), modified DNAs, modified RNAs, and other synthetic oligomers/polymers bearing natural bases, unnatural bases, or other units that interact with the target in a sequence-specific manner, such as polyacrylate analogues of nucleic acids (for example, PNA analogs), and nucleobase-containing polymers with a polyester, polyvinyl, or polyamide backbone (see, e.g., Zhou et al., J Am Chem Soc., 137(28): 8920-8923, 2015, incorporated herein by reference in its entirety).

Deoxyribozymes (DNAzymes): DNAzymes are DNA molecules capable of catalyzing specific chemical reactions. DNAzymes may catalyze nucleic acid cleavage (such as oxidative cleavage or hydrolytic cleavage, for example, phosphodiester hydrolytic cleavage), nucleoside excision, phosphorylation (or de-phosphorylation), ligation, or other reactions. DNAzymes typically include at least two binding domains that specifically bind to target sequence and a catalytic domain with enzymatic activity. DNAzymes may or may not have one or more unnatural chemical modifications on the nitrogenous bases and/or backbone thereof.

Double-strand breaks (in DNA): Breaks in which both strands of the double helix are severed. Multiple mechanisms are available for repair of double-strand breaks, including non-homologous end joining (NHEJ), microhomology-mediated end joining (MMEJ), and homologous recombination (HR, an example of homology-directed repair).

Hybridize: A process where a nucleic acid (such as a single-stranded nucleic acid) molecule binds (or anneals) to a complementary nucleic acid in a sequence-specific manner due to complementarity. Changing physiological conditions can alter hybridization. For example, increasing the surrounding temperature can cause two or more hybridized nucleic acids to separate, such as into single strands of nucleic acid. These strands are complementary to each other but may also be complementary to other sequences present in their surroundings. In some examples, lowering the surrounding temperature allows the single-strand molecules to anneal or hybridize to each other.

Inhibiting or treating a disease: Inhibiting the full development of a disease or condition, for example, in a subject who is at risk for a disease. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Peptide nucleic acid (PNA): An artificially synthesized polymer, similar to DNA or RNA, with natural nitrogenous bases (such as A, T, C, G, or U) or unnatural nitrogenous bases (such as pseudoisocytosine (Egholm et al., Nucleic Acids Res. 23(2), 217-222, 1995, incorporated herein by reference in its entirety) and G-clamp (Rapireddy et al., Biochemistry, 50(19), 3913-3918, 2011, incorporated herein by reference in its entirety)). PNA monomers can be linked through amide (—(C═O)—NH—) linkages. In some examples, a PNA backbone can be composed of repeating N-(2-aminoethyl)-glycine units linked by amides. Purine and pyrimidine bases can be linked to the backbone by a methylene bridge (—CH₂—) and a carbonyl group (—(C═O)—). In examples, PNAs can invade dsDNA, such as to form a triplex. Examples of PNAs include bis-PNA, pseudo-complementary PNA (pc-PNA), tail-clamp PNA (tc-PNA), and/or γ-PNA. Analogs of PNAs are included, such as polyacrylate nucleic acid analogs and/or nucleobase-containing polymers with polyester, polyvinyl, or polyamide backbones.

Single-stranded nucleic acid: A nucleic acid that only includes a single polymer strand (e.g., the nucleic acid polymer strand does not form non-covalent bonds with another nucleic acid polymer), such as single-stranded DNA (ssDNA). The nucleic acid molecule can be single-stranded in full (e.g., ssDNA formed through melting a double-stranded DNA molecule) or in part (e.g., a ssDNA region formed through damage and/or enzymatic activity).

Single-stranded assisting DNA (saDNA): A ssDNA molecule that acts as a catalytic nucleic acid-assisting reagent. The saDNA may include modified oligonucleotides or may be comprised entirely of DNA. The saDNA molecule is used to invade double-stranded DNA, similar to other nucleic acid-assisting reagents.

Specific binding: Binding of an agent substantially or preferentially only to a defined target such as a defined oligonucleotide, DNA, RNA, or portion thereof. Thus, a nucleic acid-specific binding agent binds substantially only to a defined nucleic acid, (such as a target site or sequence in a target nucleic acid) and does not substantially bind to any other nucleic acid. In some examples, specific binding includes the hybridization of one nucleic acid molecule to another. For example, a nucleic acid molecule specifically binds another nucleic acid molecule if a sufficient amount of the nucleic acid molecule forms base pairs or is hybridized to its target nucleic acid molecule to permit detection of that binding (such as at least 90%, 91%, 92%, 93%, 94%, 95%, 96%, 97%, 98%, 99%, or 100% complementary).

Target site: A target site is a located on a specific nucleic acid (also referred to herein as a “target nucleic acid”) and is the site of a desired activity, such as phosphodiester cleavage. Other desired activities include oxidative cleavage, nucleoside excision, phosphorylation, de-phosphorylation, and/or ligation. The target site varies based depending on the desired outcome and can include one or more specific nucleotides, nucleotide bonds (such as phosphodiester bonds), and/or modifications (such as phosphorylation, acetylation, or methylation) in a gene or sequence of interest for genetic engineering.

Vector: A nucleic acid molecule as introduced into a host cell, thereby producing a transformed, transfected, or transduced host cell. A vector may include nucleic acid sequences that permit it to replicate in the host cell, such as an origin of replication. A vector may also include one or more therapeutic genes and/or selectable marker genes and other genetic elements known in the art. A vector can transduce, transform, or transfect a cell, thereby causing the cell to synthesize nucleic acids and/or proteins other than those native to the cell. A vector may optionally be supplemented with materials to aid in achieving its entry into the cell, such as a viral particle, liposome, protein coating, or the like. A vector can be a viral vector.

### II. Overview of Several Embodiments

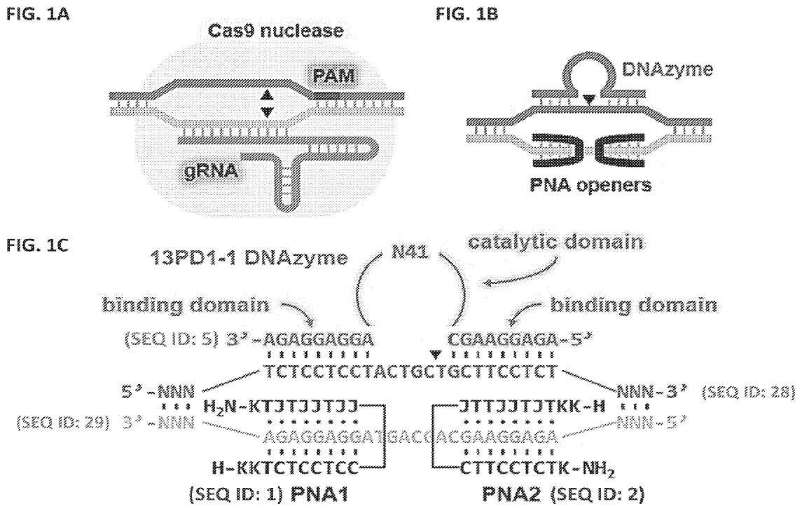

Genetic engineering systems and methods are disclosed herein. In some embodiments, the systems (which include peptide nucleic acid-assisted dsDNA nicking by DNAzymes (PANDA) systems and ssDNA-assisted double-stranded DNA nicking by DNAzymes (DANDA) systems) provide alternatives to protein restriction enzymes, or CRISPR/Cas genetic engineering systems. In prior systems, where CRISPR/Cas is used to induce double strand break (DSB), sequence specificity is provided by sgRNA, and cleavage activity provided by a Cas9 endonuclease (FIG. 1A; Doudna and Charpentier, Science, 346(6213), 1258096, 2014). In contrast, the systems and methods herein include a catalytic nucleic acid, such as a DNAzyme (also known as deoxyribozyme or catalytic DNA) that can catalyze a cleavage reaction on single-stranded DNA (ssDNA) (Xiao et al., Nucleic Acids Res., 40(4), 1778-1786, 2011). The DNAzymes described herein include programmable binding domains that specifically recognize a target substrate sequence and a conserved catalytic domain that provides DNA cleavage activity. Compared with protein enzymes, DNAzymes can be engineered to specifically recognize a user-defined sequence by simply changing the binding domain sequences. Further, in some embodiments, the catalytic activity can be fine-tuned by in vitro selection for higher reactivity and specificity.

Previously developed DNAzymes have not accepted dsDNA as a substrate because the binding domains require Watson-Crick base pairing to bind the substrate, whereas all the bases are already paired in dsDNA. As described herein, to enable DNAzyme activity on dsDNA, catalytic nucleic acid-assisting reagents are used. In some examples, the catalytic nucleic acid-assisting reagents include peptide nucleic acid (PNA). PNA strand invasion is used to open dsDNA and expose unpaired bases for DNAzymes to bind. In one embodiment, bis-PNA, which strongly binds dsDNA through Watson-Crick and Hoogsteen base paring, is used to invade dsDNA. A ssDNA-cleaving DNAzyme can bind and cleave PNA-invaded dsDNA. In other embodiments, γ-PNA is used to invade double-stranded DNA. Bases exposed by PNA invasion can be bound by the DNAzyme, which can, thus, accept dsDNA as its substrate and cleave the exposed strand, generating a nick on the dsDNA. In other examples, the catalytic nucleic acid-assisting reagents include single-stranded assisting DNA (saDNA). saDNA strand displacement is used to open dsDNA and expose unpaired bases for DNAzymes to bind. Bases exposed by saDNA strand displacement can be bound by the DNAzyme, which can, thus, accept dsDNA as its substrate and cleave the exposed strand, generating a nick on the dsDNA.

In some embodiments, two DNAzymes cleave both strands of the dsDNA (double-nicking), and a site-specific double strand break (DSB) is generated. For example, one DNAzyme can be used to cleave one strand of the dsDNA, while the other DNAzyme can be used to cleave the complementary DNA strand, resulting in the double-nicking or site-specific DSB. Unlike other gene-editing systems, such as CRISPR/Cas, both the DNAzyme and the catalytic nucleic acid-assisting reagent provide sequence-specificity independently, which should significantly reduce off-target effects. However, the systems and methods herein retain the ability to generate nicks or DSBs at user-defined sites. Thus, a variety of downstream genetic engineering applications are possible. For example, in some embodiments, the systems and methods herein may then be followed by a conventional ligation, such as using a DNA ligase, to ligate a foreign DNA to the target and generate a recombinant DNA in vitro. In other embodiments, the systems and methods herein may take advantage of subsequent cellular DNA repair machineries, such as non-homologous end joining and/or homology directed repair, to preform genetic engineering or gene editing, such as by generating mutations or repairing a mutation. Other genetic editing systems and methods are possible, such as using bis-PNAs alone or with γ-PNAs or using ssDNA-cleaving DNAzymes alone or with other catalytic nucleic acids (for example, DNA-cleaving ribozymes, base excision DNAzymes, and ligation DNAzymes).

Thus, the systems and methods herein facilitate genetic engineering with higher precision and less off-target effects, for example, as compared with CRISPR/Cas system, which produces significant genome-wide off-target effects induced by non-specific cleavage (Fu et al., Nat. Biotechnol., 31(9): 822-826, 2013). For example, in previous gene-editing systems, such as CRISPR/Cas, only one programmable element (such as guide RNA) is the guarantee of sequence specificity. In the systems and methods herein, a targeting sequence is double-checked by a catalytic nucleic acid (e.g., DNAzyme) with target-specific binding domains and a catalytic nucleic acid-assisting reagent (e.g., PNA or saDNA), as both feature high sequence selectivity.

Furthermore, catalytic nucleic acids can be engineered by in vitro selection (compared with directed evolution of protein enzymes). For example, many DNAzymes have been identified using in vitro selection to edit ssDNA, including, but not limited to, cleavage (such as oxidative cleavage or hydrolytic cleavage, for example, phosphodiester hydrolytic cleavage), nucleoside excision, phosphorylation (or de-phosphorylation), and ligation. However, engineering ssDNA has limited applications, as the major form of DNA in cells is dsDNA. Thus, the systems and methods herein expand the substrate scope of catalytic nucleic acids to dsDNA. Therefore, the benefits of catalytic nucleic acids, such as high sequence recognition specificity, single-base-level reaction site specificity, customizability, stability, and low cost, can be used for genetic engineering.

### III. Systems and Kits

Systems and kits for genetic engineering are disclosed herein. In some embodiments, the systems and kits include one or more catalytic nucleic acids and one or more catalytic nucleic acid-assisting reagents. In some embodiments, the one or more catalytic nucleic acids include one or more catalytic domains and one or more binding domains. Further, in some embodiments, the one or more catalytic nucleic acid-assisting reagents include a sequence that specifically binds a sequence that is complementary to and/or identical to a sequence flanking a target site and/or within the target site. Generally, a target site is located on a target nucleic acid, and the target site varies, based depending on the desired outcome, such as a gene or sequence of interest for genetic engineering.

A variety of catalytic nucleic acids can be included in the systems disclosed herein. In examples, the systems and kits herein include one or more catalytic nucleic acids, for example, 1, 2, 3, 4, 5, 6, 7, 8, 9, 10, 20, 30, 40, 50, 75, or 100, or about 1-2, 1-5, 10-20, 10-50, or 25-100, or 1 or 2 catalytic nucleic acids (such as DNAzymes), which may be the same or different. In some embodiments, the catalytic nucleic acids include cleavage (such as oxidative or hydrolytic cleavage, for example, phosphodiester hydrolytic cleavage), nucleoside excision, phosphorylation, de-phosphorylation, and/or ligation activity. Thus, the catalytic nucleic acids can include one or more catalytic domains (such as 1, 2, 3, 4, 5, 6, 7, 8, 9, or 10 or about 1-2, 1-5, or 1-10 catalytic domains) with cleavage (such as oxidative or hydrolytic cleavage, for example, phosphodiester hydrolytic cleavage), nucleoside excision, phosphorylation, de-phosphorylation, and/or ligation activity. In specific, non-limiting examples, the catalytic nucleic acid and/or catalytic domain includes phosphodiester hydrolytic cleavage activity, for example, nicking dsDNA or cleaving ssDNA phosphodiester bonds. In specific, non-limiting examples, the one or more catalytic nucleic acids include DNAzymes. In specific, non-limiting examples, the catalytic domains include a 13PD1 catalytic domain sequence, such as a sequence including residues 10-50 of SEQ ID NO: 5 or a 13PB2 catalytic domain sequence, such as a sequence including residues 10-50 of SEQ ID NO: 37, or a I-R3 catalytic domain sequence, such as a sequence including residues 10-19 of SEQ ID NO: 54.

Further, the catalytic nucleic acids disclosed herein also include one or more binding domains that specifically bind a target nucleic acid. For example, the catalytic nucleic acids can include 1, 2, 3, 4, 5, 6, 7, 8, 9, or 10 or about 1-2, 1-5, or 1-10 binding domains. In some embodiments, the catalytic nucleic acid includes one binding domain. In other embodiments, the catalytic nucleic acid includes at least two binding domains; for example, the catalytic nucleic acids can include at least a first and a second binding domain. Where the catalytic nucleic acids include at least two binding domains, the binding domains can vary in distance relative to their respective binding to sequences on a target nucleic acid or a nucleic acid complementary to the target nucleic acid. In other words, the binding domains can bind a target nucleic acid (or nucleic acid complementary thereto) at varying positions relative to one another. For example, the binding domains can bind at the target nucleic acid (or nucleic acid complementary thereto) within at least 0, 1, 2, 3, 4, 5, 6, 7, 8, 9, 10, 12, or 15 or about 0-1, 0-2, 0-5, 0-8, 0-10, 0-12, or 0-15 or about 5-6 nucleotides of each other. The binding domains can also bind a target nucleic acid (or nucleic acid complementary thereto) at varying positions relative to a target site. For example, the binding domains can bind at the target nucleic acid (or nucleic acid complementary thereto) within at least (−)15, (−)12, (−)10, (−)9, (−)8, (−)7, (−)6, (−)5, (−)4, (−)3, (−)2, (−)1, 0, 1, 2, 3, 4, 5, 6, 7, 8, 9, 10, 12, or 15 or about (−)15-15, (−)12-12, (−)10-10, (−)10-0, (−)8-0, (−)5-0, (−)2-0, (−)1-0, 0-2, 0-5, 0-8, or 0-10 or about (−)5 or 1 nucleotides from the target site, wherein sites with negative values (−) are 5′ of the target site, and sites with positive values are 3′ of the target site. In specific, non-limiting examples, the binding domains include a sequence that is complementary to the sequences flanking a target site. For example, the binding domains can include a first binding domain that specifically binds a sequence flanking the target site (e.g., 5′ to the target site) and a second binding domain that specifically binds a sequence flanking the target site on the opposite side compared with the first binding domain (e.g., 3′ to the target site).

A variety of binding domains can be included. The sequences of the binding domains will depend on the target nucleic acid(s). For example, the binding domains can include sequences that specifically bind to sequences flanking and/or within a target site (such as a site at which a specific reaction, such as cleavage, is desired). Thus, in some examples, the one or more binding domains include a sequence complementary to and/or identical to a sequence flanking the target site and/or within the target site. In specific, non-limiting examples, the systems and kits herein include a first binding domain specifically binds a sequence flanking the target site (e.g., 5′ of the target site), and a second binding domain specifically binds a sequence flanking the target site on the opposite side compared with the first binding domain (e.g., 3′ of the target site).

Binding domain sequences can also vary in size and melting or hybridization (annealing) temperature. In some examples, the binding domains are at least 5, 6, 7, 8, 9, 10, 11, 12, 13, 14, 15, 20, 25, 30, 35, 40, or 45, 5-50, 5-40, 5-30, 5-20, 5-15, or 7-12, or about 9 nucleotides long. Further, in some examples, binding is measured in melting temperature and can include at least about 4° C., 10° C., 15° C., 20° C., 25° C., 30° C. 32° C., 36° C., 37° C., 38° C., 40° C., 41° C., 42° C., 45° C., 50° C., 55° C., 60° C., 65° C., 70° C., 75° C., 80° C., 90° C., or 95° C. or about 4-90° C., 15-50° C., 20-40° C., 32° C.-42° C., 36° C.-42° C., 38° C.-42° C., 41° C.-42° C., 37° C.-95° C., 37° C.-60° C., or 40° C.-60° C. A person of skill in the art can extrapolate hybridization temperatures based on a variety of conditions, such as salt type and concentration (such as such as Na⁺ and/or Mg²⁺ salts; see, e.g., Tan and Chen, Biophys J., 90(4): 1175-1190, 2006, incorporated by reference herein in its entirety).

Exemplary sequences of catalytic nucleic acids (such as DNAzymes) include the following, in which the binding domains are underlined:

13PD1-1:
(SEQ ID NO: 5)
5′AGAGGAAGCTATACCGGGCAACTATTGCCTCGTCATCGCTATTTTCTG
CGAGGAGGAGA
13PD1-2:
(SEQ ID NO: 6)
5′AAGAAAGGCTATACCGGGCAACTATTGCCTCGTCATCGCTATTTTCTG
CGAGAAGAGAA
13PB2-1:
(SEQ ID NO: 37)
5′GGGTGGTCACTATGACACTTATTATAAATATGCTAGTAACGGATAGGT
TGAGGGTGGGC
I-R3-1:
(SEQ ID NO: 54)
5′CAGAGGAAGTAGTTGAGCTGGAGGAGAC
I-R3-2:
(SEQ ID NO: 55)
5′CAAGAAAGGTAGTTGAGCTGAAGAGAAC
I-R3-1i:
(SEQ ID NO: 56)
5′CAGAGGAAGATCAACTCGAGGAGGAGAC
I-R3-2i:
(SEQ ID NO: 57)
5′CAAGAAAGGATCAACTCGAGAAGAGAAC

A variety of other catalytic nucleic acid-assisting reagents are also included in the systems provided herein. Exemplary catalytic nucleic acid-assisting reagents include peptide nucleic acids (PNAs) or analogs thereof, RNAs, DNAs (such as ssDNAs), locked nucleic acids (LNAs), and other natural or unnatural polymers, for example, polyacrylate analogues of nucleic acids, and nucleobase-containing polymers with a polyester, polyvinyl, or polyamide backbone (see, e.g., Zhou et al., J Am Chem Soc., 137(28): 8920-8923, 2015, incorporated herein by reference in its entirety). Examples of PNAs include bis-PNA, pseudo-complementary PNA (pc-PNA), tail-clamp PNA (tc-PNA), and/or γ-PNA. Examples of PNA analogs include polyacrylate nucleic acid analogs and/or nucleobase-containing polymers with a polyester, polyvinyl, and/or polyamide backbones.

In specific, non-limiting examples, the catalytic nucleic acid-assisting reagents include PNAs or analogs thereof. The PNAs can include chemical modifications; for example, chemical modifications of one or more bases (e.g., a pseudoisocytosine) or backbones (e.g., methyls at the γ-position) can be included. For example, amino acid residues (e.g., a lysine) and small molecules (e.g., a eg₁ linker, or a cyanine fluorophore) can be added to terminus or inserted between two PNA sequences that are complementary to a sequence (such as one that is complementary to a sequence flanking a target site and/or the target site sequence). The PNA sequence will vary depending on the target nucleic acid. For example, where a DNAzyme is engineered to target a specific site in a dsDNA, a PNA can specifically bind a sequence that is complementary to a sequence flanking a target site and/or the target site sequence.

The catalytic nucleic acid-assisting reagents (such as PNAs or saDNAs) will vary depending on the target nucleic acid. For example, where a catalytic nucleic acid (such as a DNAzyme) is engineered to target a specific site (such as in a dsDNA), the catalytic nucleic acid-assisting reagents (such as PNAs or saDNAs) can include sequences that specifically bind a sequence that is complementary to and/or identical to a sequence flanking the target site and/or within the target site. For example, the one or more catalytic nucleic acid-assisting reagents can include sequences that form a Watson-Crick and/or Hoogsteen base pairs with a sequence that is complementary to and/or identical to a sequence flanking the target site and/or within the target site. In specific, non-limiting examples, the one or more catalytic nucleic acid-assisting reagents can include sequences that form a triple helix with a sequence complementary to the sequence flanking a target site and/or the target site sequence. Therefore, in some examples, the catalytic nucleic acid-assisting reagents (such as PNAs) are designed to invade a dsDNA and form a triplex or duplex with one of the dsDNA strands, effectively opening the dsDNA.

Exemplary sequences included in catalytic nucleic acid-assisting reagents (such as PNAs) are shown below:

PNA1:
(SEQ ID NO: 1)
H-KK-TCTCCTCC-(eg₁)₃-JJTJJTJT-K-NH₂
PNA2:
(SEQ ID NO: 2)
H-KK-TJTJJTTJ-(eg₁)₃-CTTCCTCT-K-NH₂
PNA3:
(SEQ ID NO: 3)
H-KK-TTJTTTJJ-(eg₁)₃-CCTTTCTT-K-NH₂
PNA4:
(SEQ ID NO: 4)
H-KK-TTCTCTTC-(eg₁)₃-JTTJTJTT-K-NH₂
γ-PNA1:
(SEQ ID NO: 36)
H-KKK-GCCCACCCTCGTGACCA-KKK-propargylglycine-NH₂
Y-PNA2:
(SEQ ID NO: 59)
H-KKK-AGCACTGCACGCCGTAG-KKK-propargylglycine-NH₂
(N- to C-terminus, K: lysine, J: pseudoisocytosine,
eg₁: 8-amino-3,6-dioxaoctanoyl)

In specific, non-limiting examples, the genetic engineering system can include one or more DNAzymes, comprising a catalytic domain, comprising residues 10-50 of SEQ ID NO: 5, and two binding domains, wherein a first binding domain specifically binds a sequence flanking the target site, and a second binding domain specifically binds a sequence flanking the target site on the opposite side compared with the first binding domain, and one or more peptide nucleic acids (PNAs), comprising a sequence that specifically binds and forms a triple helix with a sequence that identical to and/or complementary to a sequence within and/or flanking a target site.

In some embodiments, the system includes a 13PB2 catalytic domain sequence, such as a nucleic acid sequence including residues 10-50 of SEQ ID NO: 37, and one or more peptide nucleic acids (PNAs). In one example, the PNAs can include a γ-PNA sequence (e.g., H-KKK-GCCCACCCTCGTGACCA-KKK—NH₂ (SEQ ID NO: 36)) that specifically binds and forms a double helix with a sequence that is complementary to a sequence flanking a target site and/or the target site sequence. γ-PNA exhibits enhanced DNA binding properties such that target site sequence requirements are less stringent for γ-PNA and 13PB2, resulting in an increased catalytic efficacy.

To enable DNAzyme activity on dsDNA in the PANDA system, PNA (or PNA analog) invasion is used to open dsDNA and expose unpaired bases for DNAzymes to bind. In contrast, in the DANDA system, to enable DNAzyme activity on dsDNA, ssDNA-assisted strand invasion is used to open dsDNA and expose unpaired bases for DNAzymes to bind. Thus, in both systems, strand invasion (e.g., by PNA or saDNA) allows the DNAzyme to bind (and ultimately cleave) target DNA.

In some embodiments, reaction conditions for the PANDA system include a pH of 6-8, low or no salts, and 4° C.-40° C., ensuring specific binding between catalytic nucleic acid-assisting reagents (e.g., bis-PNA and/or γ-PNA) and the target nucleic acid. In one example, conditions sufficient for specific binding between bis-PNA or γ-PNA is pH 6.8-7.5, 0.5-2 mM Zn²⁺ at 20-37° C., for use with the DNAzyme I-R3. In some non-limiting examples, the conditions sufficient for DNAzyme activity can include a pH of 7.5, 1 mM ZnCl₂, 20 mM MnCl₂, and 25° C. In further examples, the target nucleic acid can be contacted with the one or more PNAs for at least 20 minutes. In further examples, a PNA-bound target nucleic acid can be contacted with the DNAzyme for at least 40 minutes (such as at least 40 minutes, 1 hour, 2 hours, 4 hours, 6 hours, 8 hours, or overnight). In specific, non-limiting examples, the one or more DNAzymes and the one or more PNAs can include a ratio of one PNA to one DNAzyme or two PNAs to one DNAzyme. including 13PD1 or 13PB2.

In some embodiments, reaction conditions for the ssDNA-Assisted double-stranded DNA Nicking by DNAzymes (DANDA) system include pH 6-8, high salt concentration, and elevated incubation temperatures (e.g., 40° C.-95° C.). These conditions ensure specific binding between catalytic nucleic acid-assisting reagents (e.g., saDNAs) and the target nucleic acids. In other embodiments, reaction conditions sufficient for optimized activity of DANDA include 630 mM Na⁺, 2.5 mM Mg²⁺, pH 7.5, and 75° C. In some embodiments, conditions sufficient for enhancing saDNA strand invasion of the target include incubation at 60-80° C. In other embodiments, conditions sufficient for enhancing saDNA strand invasion of the target include incubation at 4-98° C. In further examples, the target nucleic acid can be contacted with the one or more saDNAs for at least 5 minutes. In other embodiments, extension of the saDNA to at least 50 nucleotides enhances saDNA strand invasion of the target. In still other embodiments, extension of the saDNA to at least 70 nucleotides enhances saDNA strand invasion of the target.

In some embodiments, saDNA (e.g., DANDA system) activity assay preparation includes a first saDNA invasion step, followed by a second DNAzyme cleavage reaction step. In some embodiments, reaction conditions for the first saDNA invasion step include: 10 nM target DNA, 10 μM each saDNA, 630 mM NaCl, 2.5 mM MgCl₂, 10 mM MOPS buffer at pH 7.5 (indicated as concentrations in invasion reaction), heating to 75° C., and slowly cooling to room temperature. In some embodiments, reaction conditions for the second DNAzyme cleavage reaction step include: 1 mM ZnCl₂, 20 mM MnCl₂, 10 mM MgCl₂, 10 μM each DNAzyme, 10 mM MOPS buffer at pH 7.5 (indicated as final concentration), and incubation at room temperature overnight.

Further disclosed herein are kits, such as kits that include one or more catalytic nucleic acids (for example, including one or more catalytic domains and one or more binding domains) and one or more catalytic nucleic acid-assisting reagents. In some embodiments, the catalytic nucleic acids include a catalytic domain with DNA cleavage activity, such as DNAzymes (for example, ssDNA-cleaving DNAzymes). In specific, non-limiting examples, the DNAzymes include a 13PD1 catalytic domain sequence, such as residues 10-50 of SEQ ID NO: 5. In another example, the DNAzymes include 13PB2-1 (e.g., 5′ GGGTGGTCACTATGACACTTATTATAAATATGCTAGTAACGGATAGGTTGAGGGTGGG C (SEQ ID NO: 37)). In some embodiments, the catalytic nucleic acids include one or more binding domains with a sequence that is complementary to a sequence within and/or flanking the target site. In some embodiments, the catalytic nucleic acid-assisting reagents (such as PNAs, saDNAs, analogs thereof, and/or RNAs) include sequences that specifically bind a sequence that is complementary to and/or identical to a sequence flanking the target site and/or within the target site. In some examples, the catalytic nucleic acid-assisting reagents include a sequence that specifically binds a sequence that is identical to and/or complementary to a sequence within and/or flanking a target site.

Also contemplated herein are additional reagents, such as reagents for use with the systems provided herein, such as solutions, buffers, and/or salts. In some embodiments, the kits disclosed herein include reagents for use in providing specific binding between the target nucleic acid and the one or more catalytic nucleic acid-assisting reagents. For example, where the catalytic nucleic acid-assisting reagents include PNAs, the kits can include binding solutions (such as buffered solutions) or reagents therefor with a pH of 5.5-8.5 (such as a pH at least about 5.5, 5.8, 6, 6.2, 6.5, 6.8, 7, 7.2, 7.5, 7.8, 8, 8.2, or 8.5 or about 5.5-7.5, 6-8, 6-8.5, 7-8, 7.2-7.8, or 7.2-7.5 or about 7.5) or low or no metal ions (such as monovalent metal ions, for example, in a salt and/or at least about 0, 2, 5, 10, 15, or 20 mM or about 0-20, 0-2, 0-5, 0-10, 5-20, 5-15, or 5-10 mM or about 0-10 mM metal ions). For example, where the catalytic nucleic acid-assisting reagents are saDNA, the kits can include binding solutions (such as buffered solutions) or reagents therefor with a pH of about 5.5-8.5 (such as a pH at least about 5.5, 5.8, 6, 6.2, 6.5, 6.8, 7, 7.2, 7.5, 7.8, 8, 8.2, or 8.5 or about 5.5-7.5, 6-8, 6-8.5, 7-8, 7.2-7.8, or 7.2-7.5 or about 6.8-7.5) and high metal ions (such as monovalent metal ions, for example, in a salt and/or at least about 100-800 mM or about 100-250, 200-400, 350-500, 400-600, 500-700, or 600-800 mM metal ions).

In some embodiments, the kits disclosed herein can include reagents for use with the provided catalytic nucleic acids. For example, where the catalytic nucleic acids include DNAzymes, the kits can include binding solutions (such as buffered solutions) or reagents therefor with a pH of 5.5-8.5 (such as a pH at least about 5.5, 5.8, 6, 6.2, 6.5, 6.8, 7, 7.2, 7.5, 7.8, 8, 8.2, or 8.5 or about 5.5-7.5, 6-8, 6-8.5, 7-8, 7.2-7.8, or 7.2-7.5 or about 7.5) and/or metal ion concentrations sufficient for the DNAzyme activity (such as zinc or manganese metal ions, such as in salt form, for example, ZnCl₂ and/or MnCl₂ and/or at least about 0.5, 0.75, 1, 1.5, or 2 mM or 0.5-2 or 1-1.5 mM or about 1 mM Zn and/or at least about 5, 7.5, 10, 12, 15, 18, 20, or 25 mM or about 5-25, 10-20, or 15-20 mM or about 20 mM MnCl₂; other ions and salts can be included, such as MgCl₂ at about 2-20 mM, such as about 10 mM, and/or NaCl at about 15-50 mM, such as about 37.5 mM).

Kits contemplated herein can further include a variety of catalytic nucleic acids (such as DNAzymes) and/or catalytic nucleic acid-assisting reagents (such as PNAs or saDNAs). For example, 1, 2, 3, 4, 5, 6, 7, 8, 9, 10, 20, 30, 40, 50, 75, or 100, or about 1-2, 1-5, 10-20, 10-50, or 25-100, or about 1 or 2 catalytic nucleic acids can be included. Further, the one or more catalytic nucleic acids and the one or more catalytic nucleic acid-assisting reagents can be included at a variety of ratios, such as at least 1:1, 1:2, 1:3, 1:4, 1:5, or 1:10, or about 1:1-1:5 or 1:1-1:2 or about 1:2 catalytic nucleic acids (such as DNAzymes) to catalytic nucleic acid-assisting reagents (such as PNAs).

A variety of catalytic nucleic acid-assisting reagents, DNAzymes, modified oligonucleotides, and nonnative backbones are contemplated by the present invention. As described above, various RNAs, DNAs (such as ssDNAs), peptide nucleic acids (PNAs), locked nucleic acids (LNAs), and other natural or unnatural polymers are contemplated. For example, polyacrylate analogues of nucleic acids may be used, in addition to nucleobase-containing polymers with a polyester, polyvinyl, or polyamide backbone. Nonnative backbones may include bifacial peptide nucleic acids displaying melamine, for example. Notably, polyacrylate backbones displaying melamine can triplex hybridize efficiently with native bases and nucleic acids, bridging various native and artificial architectures. Additional nucleic acid modifications may be made to the catalytic nucleic acids and/or catalytic nucleic acid-assisting reagents described herein, including modified riboses. In other embodiments, standard modified oligonucleotides or nucleic acids modifications may be used such as cross-linking, methylation, phosphorothioate incorporation, encapsulation in lipid nanoparticles, and the like.

In another embodiment, in vitro selection may be used to obtain optimized catalytic nucleic acid and/or catalytic nucleic acid-assisting reagent variants. In some embodiments, enhanced catalytic rates are achieved and/or target specificity is improved. In other embodiments, aptazyme functionality may be incorporated into the PANDA system. For example, stem or loop regions of dsDNA proximal to the DNAzyme catalytic core may be engineered to bind effector molecules, including small molecules, proteins, or other nucleic acids such that a nucleic acid switch is formed. In other examples, a DNAzyme and/or substrate strand tertiary contact is engineered via in vitro evolution such that binding of an effector molecule (e.g., a protein, small molecule, or the like) upstream of the tertiary contact biases the DNAzyme catalytic core to favor cleaved product formation. In this context, effector molecules selectively bind to an effector-binding region of the DNAzyme and regulate its biological activity. Thus, effector molecules perform a targeting function, acting as ligands that can increase or decrease enzyme activity, gene expression, or cell signaling. Effector molecules can also directly regulate the activity of some DNA and/or RNA molecules.

In some embodiments, the catalytic-nucleic acid-assisting reagent and the DNAzyme are engineered as one contiguous strand, rather than comprising two or more separate elements. In some embodiments, click chemistry facilitates conjugation of a DNAzyme and a catalytic-nucleic acid-assisting reagent. For example, propargylglycine may be used to conjugate PNA and a DNAzyme. In another example, a discontinuous DNAzyme catalytic core is engineered wherein the enzyme strand loop region is extended by hundreds of base pairs. In some embodiments, a discontinuous PANDA or DANDA system may be engineered such that the intervening region binds to effector molecules, allowing strand hybridization and DNAzyme cleavage activity only upon binding of the effector molecule.

In other embodiments, the catalytic nucleic acid-assisting reagent may be modified to incorporate non-native moieties. In some embodiments, the catalytic nucleic acid-assisting reagent is comprised entirely of synthetic molecules. In other embodiments, monovalent or divalent ion binding sites are modified in the DNAzyme and/or the catalytic nucleic acid-assisting reagent to modulate activity.

Importantly, when the catalytic nucleic acid-assisting reagent comprises a PNA, said PNAs can include other positively charged moieties to increase the solubility of the PNA and increase the affinity of the PNA for duplex DNA. Commonly used positively charged moieties include the amino acids lysine and arginine, although other positively charged moieties may also be useful.

Lysine and arginine residues can be added to a bis-PNA linker or can be added to the carboxy or the N-terminus of a PNA strand. Common modifications to PNA are discussed in Sugiyama and Kittaka, Molecules, 18:287-310 (2013) and Sahu, et al., J. Org. Chem., 76, 5614-5627 (2011), each of which are specifically incorporated by reference in their entireties, and include, but are not limited to, incorporation of charged amino acid residues, such as lysine at the termini or in the interior part of the oligomer; inclusion of polar groups in the backbone, carboxymethylene bridge, and in the nucleobases; chiral PNAs bearing substituents on the original N-(2-aminoethyl)glycine backbone; replacement of the original aminoethylglycyl backbone skeleton with a negatively-charged scaffold; conjugation of high molecular weight polyethylene glycol (PEG) to one of the termini; fusion of PNA to DNA to generate a chimeric oligomer, redesign of the backbone architecture, conjugation of PNA to DNA or RNA. These modifications improve solubility but may result in reduced binding affinity and/or sequence specificity.

One class of γ substitution, is miniPEG, but other residues and side chains can be considered, and even mixed substitutions can be used to tune the properties of the above described PANDA systems. “MiniPEG” and “MP” refers to diethylene glycol. MiniPEG-containing yPNAs are conformationally preorganized PNAs that exhibit superior hybridization properties and water solubility as compared to the original PNA design and other chiral γ-PNAs. γ-PNAs prepared from L-amino acids adopt a right-handed helix, while those prepared from D-amino acids adopt a left-handed helix; however, only the right-handed helical γ-PNAs hybridize to DNA or RNA with high affinity and sequence selectivity. In the most preferred embodiments, some or all of the PNA monomers are miniPEG-containing γ-PNAs (Sahu, et al., J. Org. Chem., 76, 5614-5627 (2011). In the embodiments, tcPNAs are prepared wherein every other PNA monomer on the Watson-Crick binding side of the linker is a miniPEG-containing γ-PNA. Accordingly, the tail clamp side of the PNA has alternating PNA and miniPEG-containing γ-PNA monomers.

In some embodiments additional or alternative γ substitutions or other PNA chemical modifications are possible, including but limited to those described herein. Examples of γ substitution with other side chains include that of alanine, serine, threonine, cysteine, valine, leucine, isoleucine, methionine, praline, phenylalanine, tyrosine, aspartic acid, glutamic acid, asparagine, glutamine, histidine, lysine, arginine, and the derivatives thereof. The term “derivatives thereof” herein is defined as those chemical moieties that are covalently attached to these amino acid side chains, for instance, to that of serine, cysteine, threonine, tyrosine, aspartic acid, glutamic acid, asparagine, glutamine, lysine, and arginine.

Additionally, any of the PNA sequences can be modified to include guanidine-G-clamp (“G-clamp”) PNA monomer(s) to enhance PNA binding. γPNAs with substitution of cytosine by G-clamp (9-(2-guanidinoethoxy) phenoxazine), a cytosine analog that can form five H-bonds with guanine, and can also provide extra base stacking due to the expanded phenoxazine ring system and substantially increased binding affinity.

### IV. Methods of Genetic Engineering

Disclosed herein are methods of genetic engineering using the systems and kits herein. The methods can include editing a variety of nucleic acids. For example, the target nucleic acid can be RNA or DNA, such as a single- or double-stranded DNA (ssDNA or dsDNA). Further, the methods can be used in vitro, ex vivo, in vivo, or in cellulo. For example, the methods can be used for molecular cloning or to target genomic DNA, for example in a cell. Thus, the systems and kits disclosed herein can be used to treat diseases or disorders (such as in a subject). For example, the systems and kits disclosed herein can be used to edit (e.g., correct) a gene that is wholly or partially responsible for a disease or disorder. In some examples, the systems and kits disclosed herein can be used to modulate expression of one or more biological molecules, thereby treating a disease or disorder (such as in a subject).

The methods herein include contacting a target nucleic acid with one or more catalytic nucleic acid-assisting reagents as described herein (such as PNAs or analogs thereof, ssDNAs, RNAs, DNAs, LNAs, polyamides, or analogs thereof). For example, the one or more catalytic nucleic acid-assisting reagents contacted herein can include a sequence that specifically binds a sequence that is complementary to and/or identical to a sequence flanking a target site and/or within a target site. Thus, the methods can include generating a catalytic nucleic acid-assisting reagent-bound target nucleic acid (for example, where the contacting occurs under conditions sufficient for specific binding between the target nucleic acid and the one or more catalytic nucleic acid-assisting reagents). The methods further include contacting the catalytic nucleic acid-assisting reagent-bound target nucleic acid with one or more catalytic nucleic acids (for example, under conditions sufficient for catalytic nucleic acid activity). For example, the catalytic nucleic acids contacted herein can include one or more catalytic domains and one or more binding domains. In some examples, the one or more binding domains include a sequence complementary to a sequence flanking the target site. The target nucleic acid may be contacted with the one or more catalytic nucleic acid-assisting reagents and the one or more catalytic nucleic acids sequentially or concurrently.

The methods herein include contacting the target nucleic acid with catalytic nucleic acid-assisting reagents under a variety of conditions. In some embodiments, the methods include contacting the target nucleic acid with catalytic nucleic acid-assisting reagents under conditions sufficient for specific binding of the one or more catalytic nucleic acid-assisting reagents. In specific, non-limiting examples, the methods include contacting the target nucleic acid with one or more PNAs or analogs thereof. Thus, the contacting can occur under conditions suitable for specific binding between the target nucleic acid and the one or more PNAs. Other catalytic nucleic acid-assisting reagents (such as polyacrylate analogs of nucleic acids and/or nucleobase-containing polymers with polyester, polyvinyl, or polyamide backbones, for example, PNA analogs) can be used in the methods herein under similar conditions (see, for example, Zhou et al., J Am Chem Soc., 137(28): 8920-8923, 2015, incorporated herein by reference in its entirety).

A variety of conditions may be suitable for specific binding between the target nucleic acid and the one or more PNAs, such as pH, temperature, contacting or binding time, and salt concentration. For example, the conditions suitable for specific binding between a nucleic acid (such as a target nucleic acid) and one or more PNAs can include a pH of 6-8 (such as a pH at least about 6, 6.2, 6.5, 6.8, 7, 7.2, 7.5, 7.8, 8, or about 5.5-7.5, 6-8, 7-8, 7.2-7.8, or 7.2-7.5 or about 7.5), 4° C.-40° C. (such as at least about 4° C., 10° C., 15° C., 20° C., 22° C., 25° C., 28° C., 30° C., 32° C., 35° C., 37° C., or 40° C. or about 4-20° C., 10-25° C., 20° C.-37° C., 22° C.-37° C., 25-37° C., 30° C.-37° C., or 35-37° C. or about 37° C.), low or no metal ions (such as monovalent metal ions, for example, in a salt and/or at least about 0, 2, 5, 10, 15, or 20 mM or about 0-20, 0-2, 0-5, 0-10, 5-20, 5-15, or 5-10 mM or about 0-10 mM metal ions), and/or contacting or binding time of at least 15 min (such as at least 15 min, 18 min, 20 min, 25 min, 30 min, 40 min, 60 min, 1.5 hr, 2 hr, 4 hr, 6 hr, 8 hr, 10 hr, 12 hr, 18 hr, or overnight or about 15 min-overnight, 20 min-overnight, 40 min-overnight, 2 hr-overnight, 20 min-18 hr, 40 min-18 hr, 2 hr-18 hr, 6 hr-18 hr, or 8 hr-12 hr about 20 min or 2 hr).

A variety of conditions may be suitable for specific binding between the target nucleic acid and the one or more saDNAs, such as pH, temperature, contacting or binding time, and salt concentration. For example, the conditions suitable for specific binding between a nucleic acid (such as a target nucleic acid) and one or more saDNAs can include a pH of 6-8 (such as a pH at least about 6, 6.2, 6.5, 6.8, 7, 7.2, 7.5, 7.8, 8, or about 5.5-7.5, 6-8, 7-8, 7.2-7.8, or 7.2-7.5 or about 6.8-7.5), 40-95° C. (such as a temperature of at least about 40° C., 50° C., 60° C., 70° C., 75° C., 80° C., 85° C., 90° C., or 95° C.), high metal ions (such as monovalent metal ions, for example, in a salt and/or at least about 100-800 mM or about 100-250, 200-400, 350-500, 400-600, 500-700, or 600-800 mM metal ions), and/or contacting or binding time of at least 3 min, followed by slow cooling to room temperature (such as at least 3 min, 5, min, 10 min, 15 min, 18 min, 20 min, 25 min, 30 min, 40 min, 60 min, 1.5 hr, 2 hr, 4 hr, 6 hr, 8 hr, 10 hr, 12 hr, 18 hr, or overnight or about 15 min-overnight, 20 min-overnight, 40 min-overnight, 2 hr-overnight, 20 min-18 hr, 40 min-18 hr, 2 hr-18 hr, 6 hr-18 hr, or 8 hr-12 hr about 20 min or 2 hr).

The methods herein also include contacting the catalytic nucleic acid-assisting reagent-bound target nucleic acid with one or more catalytic nucleic acids under a variety of conditions. In some embodiments, the methods include contacting the catalytic nucleic acid-assisting reagent-bound target nucleic acid with one or more catalytic nucleic acids under conditions sufficient for catalytic nucleic acid activity. In specific, non-limiting examples, the one or more catalytic nucleic acids include DNAzymes. Thus, the contacting can occur under conditions sufficient for DNAzyme activity. A variety of conditions may be suitable for DNAzyme activity, such as pH, temperature, contacting or binding time, and salt concentration. For example, the conditions suitable for DNAzyme activity can include a pH of 6-8 (such as a pH at least about 6, 6.2, 6.5, 6.8, 7, 7.2, 7.5, 7.8, 8, or about 5.5-7.5, 6-8, 7-8, 7.2-7.8, or 7.2-7.5 or about 6.8-7.5), metal ion concentrations sufficient for the DNAzyme activity (such as zinc or manganese metal ions, such as in salt form, for example, ZnCl₂ and/or MnCl₂ and/or at least about 0.5, 0.75, 1, 1.5, or 2 mM or 0.5-2 or 1-1.5 mM or about 1 mM Zn and/or at least about 5, 7.5, 10, 12, 15, 18, 20, or 25 mM or about 5-25, 10-20, or 15-20 mM or about 20 mM MnCl₂; other ions and salts are possible, such as MgCl₂ at about 2-20 mM, such as about 10 mM, and/or NaCl at about 15-50 mM, such as about 37.5 mM), a temperature of about 4° C.-50° C. (such as a temperature of at least about 4° C., 10° C., 20° C., 25° C., 30° C., 35° C., 40° C., 45° C., or 50° C.), and/or an activity time of about 10 min-overnight (such as such as at least 10 minutes, 20 minutes, 30 minutes, 40 minutes, 45 minutes, 1 hour, 2 hours, 4 hours, 6 hours, 8 hours, 12 hours, 18 hours, or overnight, or about 10 minutes-2 hours, 1 hour-8 hours, 2 hours-18 hours, 40 minutes-18 hours, or 40 minutes-overnight, or about 40 minutes).

In some embodiments, reaction procedures for the PANDA system include a first PNA invasion reaction step, followed by a second DNAzyme cleavage reaction step. In some non-limiting examples, the conditions for the PNA invasion reaction step include conditions sufficient for specific binding between the PNA (e.g., bis-PNA and/or γ-PNA) and the target nucleic acid, such as a pH of about 6-8, low or no salt concentration, and about 4° C.-40° C. In one example, conditions sufficient for specific binding between the target and bis-PNA or γ-PNA is pH 7.5, low or no salt, and 20-37° C. In further examples, the target nucleic acid can be contacted with the one or more PNAs for at least 20 minutes. In some non-limiting examples, the conditions sufficient for DNAzyme activity can include a pH of 6-8, 0-2 mM ZnCl₂, 0-20 mM MnCl₂, and 20-37° C. In one example, conditions sufficient for DNAzyme activity is pH 7.5, 1 mM ZnCl₂, 20 mM MnCl₂, and 25° C., for 13PD1 or 13PB2 DNAzymes. In another example, conditions sufficient for DNAzyme activity is pH 7.5, 2 mM ZnCl₂, 0 mM MnCl₂, and 25° C., for I-R3 DNAzymes. In other examples, conditions sufficient for DNAzyme activity is the same or close to the condition used in the in vitro selection steps of the DNAzymes, such as pH 7.5, 20 mM MgCl₂, and 37° C. In further examples, a PNA-bound target nucleic acid can be contacted with the DNAzyme for at least 40 minutes (such as at least 40 minutes, 1 hour, 2 hours, 4 hours, 6 hours, 8 hours, or overnight).

In other embodiments, reaction procedures for the PANDA system include one single step for both PNA invasion and DNAzyme activity. In some non-limiting examples, the conditions for this step is pH 6-8, 0-2 mM Zn²⁺, and 4-40° C. In one example, the conditions for this step is pH 7.5, 1 mM Zn²⁺, and 37° C. In other examples, conditions sufficient for this step is the same or similar to the condition used in the in vitro selection steps of the DNAzymes, such as pH 7.5, 20 mM MgCl₂, and 37° C. In further examples, the target nucleic acid can be contacted with the PNA and the DNAzyme at the same time for at least 40 minutes (such as at least 40 minutes, 1 hour, 2 hours, 4 hours, 6 hours, 8 hours, or overnight). In a specific, non-limiting examples, the one or more DNAzymes and the one or more PNAs can include a ratio of one PNA to one DNAzyme or two PNAs to one DNAzyme.

In some embodiments, reaction procedures for the ssDNA-Assisted double-stranded DNA Nicking by DNAzymes (DANDA) system include a first saDNA invasion reaction step, followed by a second DNAzyme cleavage reaction step. In some non-limiting examples, the conditions for the saDNA invasion reaction step include conditions sufficient for specific binding between the saDNA and the target nucleic acid, such as pH of about 6-8, elevated salt concentration, and elevated incubation temperatures (e.g., 40° C.-95° C.). In one example, conditions sufficient for specific binding between the target and saDNA is pH 7.5, 630 mM Na⁺, 2.5 mM Mg²⁺, pH 7.5, and 75° C. In further examples, the target nucleic acid can be contacted with the one or more saDNA for at least 3 minutes, followed by a slow cooling step to room temperature. In some non-limiting examples, the conditions sufficient for DNAzyme activity can include a pH of 6-8, 0-2 mM ZnCl₂, 0-20 mM MnCl₂, and 20-37° C. In one example, conditions sufficient for DNAzyme activity is pH 7.5, 1 mM ZnCl₂, 20 mM MnCl₂, and 25° C., for 13PD1 or 13PB2 DNAzymes. In another example, conditions sufficient for DNAzyme activity is pH 7.5, 2 mM ZnCl₂, 0 mM MnCl₂, and 25° C., for I-R3 DNAzymes. In other examples, conditions sufficient for DNAzyme activity is the same or similar to the conditions used in the in vitro selection steps of the DNAzymes, such as pH 7.5, 20 mM MgCl₂, and 37° C. In further examples, a saDNA-bound target nucleic acid can be contacted with the DNAzyme for at least 40 minutes (such as at least 40 minutes, 1 hour, 2 hours, 4 hours, 6 hours, 8 hours, or overnight). In specific, non-limiting examples, the one or more DNAzymes and the one or more saDNAs can include a ratio of one saDNA to one DNAzyme or two saDNAs to one DNAzyme. In other embodiments, extension of the saDNA to at least 50 nucleotides enhances saDNA strand invasion of the target. In still other embodiments, extension of the saDNA to at least 70 nucleotides enhances saDNA strand invasion of the target.

In some embodiments, the reaction for the PANDA or the DANDA system can be conducted in living cells or under physiological conditions. Thus, in some examples, the buffer or reaction conditions utilized are adjusted to reflect more closely the conditions in a cell. For example, the reaction conditions may include potassium, sodium, magnesium, calcium, and/or chloride ions, and in some examples, may not include zinc. One of ordinary skill in the art can select appropriate ion concentrations depending on the physiologic systems (e.g., bacterial, yeast, or mammalian). See, e.g., Cell Biology by the Numbers, Ron Milo and Rob Phillips, Garland Science, 2015, pages 127-135.

In some non-limiting examples, the one or more DNAzymes and the one or more catalytic nucleic acid-assisting reagents (such as PNAs and/or saDNAs) can be delivered to the cells by transfection, transduction, or transformation. In one example, the delivery can include an electroporation step. In examples, the treatment of the cells after the delivery of the one or more DNAzymes and the one or more catalytic nucleic acid-assisting reagents can include the addition of supplementary metal ions to the culture media, such as Zn²⁺, Mn²⁺, or Mg²⁺ to allow DNAzyme activity. In specific, non-limiting examples, the metal ion added to the culture media is ZnCl₂ to final concentration of 0.5 mM. In other examples, the metal ion added to the culture media is the same or similar to the metal ions included in the in vitro selection steps of the DNAzymes, such as MgCl₂ to final concentration of 20 mM. In other examples, the excess amount of the DNAzymes, the catalytic-nucleic acid-assisting reagents, and/or the supplementary metal ions in the culture media can optionally be removed by washing the cells after a period of incubation, such as 0 hours, 1 hour, 2 hours, 4 hours, 6 hours, 8 hours, or overnight, for optimal cell viability.

In the methods herein, the number of catalytic nucleic acids used for genetic engineering can vary. In some embodiments, cleavages of the nucleic acid in at least one site will be desired (such as one or more target sites). Depending on the number of catalytic nucleic acid and/or number of target sites, the catalytic nucleic acid can generate single-strand or double-strand breaks on a nucleic acid (such as a dsDNA). Thus, one or more different catalytic nucleic acids can be used, including catalytic nucleic acids with one or more different activities or that recognize one or more different target nucleic acid sites. For example, 1, 2, 3, 4, 5, 6, 7, 8, 9, 10, 20, 30, 40, 50, 75, or 100, or about 1-2, 1-5, 10-20, 10-50, or 25-100, or about 1 or 2 catalytic nucleic acids can be used. Further, the one or more catalytic nucleic acids and the one or more catalytic nucleic acid-assisting reagents can be used at a variety of ratios, such as at least 1:1, 1:2, 1:3, 1:4, 1:5, or 1:10, or about 1:1-1:5 or 1:1-1:2 or about 1:2 catalytic nucleic acids to catalytic nucleic acid-assisting reagents.

### EXAMPLES

The following examples are provided to illustrate certain particular features and/or embodiments. These examples should not be construed to limit the disclosure to the particular features or embodiments described.

### Example 1: Materials and Methods

PNA oligomers were synthesized, HPLC purified by PANAGENE (Daejeon, Korea), and used as received. DNA oligonucleotides, including DNAzymes, control oligonucleotides, primers, ssDNA templates, and molecular cloning insert fragments, were all synthesized and desalted by Integrated DNA Technologies (IDT®). DNAzymes and control oligonucleotides were purified by denatured PAGE prior to use. Other oligonucleotides were used as received.

The PNA sequences used in this example are as follows (N- to C-terminus, K: lysine, J: pseudoisocytosine, eg₁: 8-amino-3,6-dioxaoctanoyl, H: free amine at the N-terminus, NH₂: amide at the C-terminus):

PNA1:
(SEQ ID NO: 1)
H-KK-TCTCCTCC-(eg₁)₃-JJTJJTJT-K-NH2
PNA2:
(SEQ ID NO: 2)
H-KK-TJTJJTTJ-(eg₁)₃-CTTCCTCT-K-NH₂
PNA3:
(SEQ ID NO: 3)
H-KK-TTJTTTJJ-(eg₁)₃-CCTTTCTT-K-NH₂
PNA4:
(SEQ ID NO: 4)
H-KK-TTCTCTTC-(eg₁)₃-JTTJTJTT-K-NH₂.

The DNAzyme sequences used in this example are as follows (with the binding domains underlined):

13PD1-1:
(SEQ ID NO: 5)
5′AGAGGAAGCTATACCGGGCAACTATTGCCTCGTCATCGCTATTTTCT
GCGAGGAGGAGA
13PD1-2:
(SEQ ID NO: 6)
5′AAGAAAGGCTATACCGGGCAACTATTGCCTCGTCATCGCTATTTTCT
GCGAGAAGAGAA

The control oligonucleotide sequences used in this example are as follows (with the binding domains underlined):

13PD1-li:
(SEQ ID NO: 7)
5′AGAGGAAGCATATGGCCCGTTGATAACGGAGCAGTAGCGATAAAAGA
CGCAGGAGGAGA
13PD1-un:
(SEQ ID NO: 8)
5′CATCTCTTCTATACCGGGCAACTATTGCCTCGTCATCGCTATTTTCT
GCGATAGTGAGT
T59:
(SEQ ID NO: 9)
5′TTTTTTTTTTTTTTTTTTTTTTTTTTTTTTTTTTTTTTTTTTTTTTT
TTTTTTTTTTTT.

The primers used in this example are as follows:

FP1:
(SEQ ID NO: 10)
5′CGGCACCTCGCTAACGGATTCG
RP1:
(SEQ ID NO: 11)
5′CAACTCGGTCGCCGCATACACG
DraIII-FP2:
(SEQ ID NO: 12)
5′TGACACACGTAGTGGTAAACAGGTGGCAAGCGTCGC
DraIII-RP2:
(SEQ ID NO: 13)
5′AGACACACTACGTGTTGTTGCATGGTCTGGTGGTGC

The templates used in this example are as follows:

Temp-NK1:
(SEQ ID NO: 14)
5′CGGCACCTCGCTAACGGATTCGCCTCAGCCGTCTCCTCCTACTGCTGC
TTCCTCTCGGAATGCTACTTCGTGTATGCGGCGACCGAGTTG
Temp-NK1m1:
(SEQ ID NO: 15)
5′CGGCACCTCGCTAACGGATTCGCCTCAGCCGTCTCGTCCTACTGCTGC
TTCCTCTCGGAATGCTACTTCGTGTATGCGGCGACCGAGTTG
Temp-NK1m2:
(SEQ ID NO: 16)
5′CGGCACCTCGCTAACGGATTCGCCTCAGCCGTCTCCTCCTACTGCTGC
TTGCTCTCGGAATGCTACTTCGTGTATGCGGCGACCGAGTTG
Temp-NK1m3:
(SEQ ID NO: 17)
5′CGGCACCTCGCTAACGGATTCGCCTCAGCCGTCTCGTCCTACTGCTGC
TTGCTCTCGGAATGCTACTTCGTGTATGCGGCGACCGAGTTG
Temp-DSB1:
(SEQ ID NO: 18)
5′CGGCACCTCGCTAACGGATTCGGAATTCATCTCCTCCTACTGCTGCTT
CCTCTATAAGAAAGGCAGCAGTAGAAGAGAAATGATCACGTGTATGCGGC
GACCGAGTTG
Temp-DSB2:
(SEQ ID NO: 19)
5′GTAAACAGGTGGCAAGCGTCGCTTATTAAAAGAAAGGCAGCAGTAGA
AGAGAAATTCTCCTCCTACTGCTGCTTCCTCTAAGCTGTGCACCACCAGA
CCATGCAACAA.

Molecular cloning insert fragments used in this example are as follows (with cohesive ends (underlined) and NheI recognition sites (underlined italic)):

pS3ins-F:
(SEQ ID NO: 20)
5′TGCCTTTCTTATAGAGGAAGCATATACCTCGCATTGGAGCTAGCTACA
TGTTGGCACTGGGCAGTAGAAGAGAAATTCTCCTCCTACTGC
pS3ins-R:
(SEQ ID NO: 21)
5′CCAGTGCCAACATGTAGCTAGCTCCAATGCGAGGTATA

Plasmids for the Peptide Nucleic Acid-Assisted dsDNA Nicking by DNAzymes (PANDA) cleavage assays were prepared as follows. Sequences containing appropriate DNAzyme, PNA, and protein nickase recognition sites were PCR-amplified from ssDNA templates and inserted to pCRII vector (Invitrogen®) by either TA cloning (Invitrogen® TA Cloning Kit) or standard restriction enzyme-based cloning (DraIII-HF and T4 DNA ligase, New England Biolabs (NEB®)) following recommended procedures provided by Invitrogen® or NEB®. NEB® 5-alpha competent E. coli (Subcloning Efficiency) (NEB®) were transformed with recombinant plasmids and miniprepped using a QIAprep® Miniprep Kit (QIAGEN®).

The corresponding primers, templates, and vectors used in plasmid preparations are as follows:
Plasmid 1: PCR fragment amplified from Temp-NK1 with FP1 and RP1, cloned to pCRII (TA cloning site)
Plasmid 1 m1: PCR fragment amplified from Temp-NK1 m1 with FP1 and RP1, cloned to pCRII (TA cloning site)
Plasmid 1m2: PCR fragment amplified from Temp-NK1m2 with FP1 and RP1, cloned to pCRII (TA cloning site)
Plasmid 1m3: PCR fragment amplified from Temp-NK1m3 with FP1 and RP1, cloned to pCRII (TA cloning site)
Plasmid 2: PCR fragment amplified from Temp-DSB1 with FP1 and RP1, cloned to pCRII (TA cloning site)
Plasmid 3: PCR fragment amplified from Temp-DSB2 with DraIII-FP2 and DraIII-RP2, digested with DraIII, cloned to plasmid 2 (DraIII site).

PANDA recognition sites on these plasmids were confirmed by Sanger sequencing as indicated below. Only forward strands are shown; J and T denote cleavage sites on forward and reverse strands, respectively, as follows:
Plasmid 1: one PANDA recognition site (PANDA-A, underlined) and one protein nickase Nb.BbvCI recognition site (underlined italic):

(SEQ ID NO: 22)
5′CAACTCGGTCGCCGCATACACGAAGTAGCATTCCGAGAGGAAGCA↑GC
AGTAGGAGGAGACGGC↓TGAGGCGAATCCGTTAGCGAGGTGCCG

Plasmid 1m1: PANDA recognition site (PANDA-A, underlined) with one base mismatched (bold):

(SEQ ID NO: 23)
5′CAACTCGGTCGCCGCATACACGAAGTAGCATTCCGAGAGGAAGCAGCA
GTAGGACGAGACGGCTGAGGCGAATCCGTTAGCGAGGTGCCG

Plasmid 1m2: PANDA recognition site (PANDA-A, underlined) with one base mismatched (bold):

(SEQ ID NO: 24)
5′CAACTCGGTCGCCGCATACACGAAGTAGCATTCCGAGAGCAAGCAGCA
GTAGGAGGAGACGGCTGAGGCGAATCCGTTAGCGAGGTGCCG

Plasmid 1m3: PANDA recognition site (PANDA-A, underlined) with two bases mismatched (bold):

(SEQ ID NO: 25)
5′CAACTCGGTCGCCGCATACACGAAGTAGCATTCCGAGAGCAAGCAGCA
GTAGGACGAGACGGCTGAGGCGAATCCGTTAGCGAGGTGCCG

Plasmid 2: two different PANDA recognition sites (PANDA-A, underlined, and PANDA-B, underlined italic):

(SEQ ID NO: 26)
5′CAACTCGGTCGCCGCATACACGTGATCATTTCTCTTCTACTGC↓TGCC
TTTCTTATAGAGGAAGCA↑GCAGTAGGAGGAGATGAATTCCGAATCCGTT
AGCGAGGTGCCG

Plasmid 3, four PANDA recognition sites (PANDA-A, underlined, and PANDA-B, underlined italic:

(SEQ ID NO: 27)
5′CAACTCGGTCGCCGCATACACGTGATCATTTCTCTTCTACTGC↓TGCC
TTTCTTATAGAGGAAGCA↑GCAGTAGGAGGAGATGAATTCCGAATCCGTT
AGCGAGGTGCCG......GTGGTAAACAGGTGGCAAGCGTCGCTTATTAA
AAGAAAGGCA↑GCAGTAGAAGAGAAATTCTCCTCCTACTGC↓TGCTTCCT
CTAAGCTGTGCACCACCAGACCATGCAACAACACGTA.

The corresponding PNA and DNAzymes for PANDA systems used in this example are as follows:

PANDA-A: PNA1, PNA2 and 13PD1-1

PANDA-B: PNA3, PNA4 and 13PD1-2.

The PANDA treatment procedures included sequential PNA invasions and DNAzyme cleavage reactions. For PNA invasion, plasmids containing the appropriate recognition sites were mixed with PNAs in 10 mM MOPS buffer at pH 7.5 to final concentrations of 10 nM plasmid and 400 nM each of PNAs. The mixture was incubated at 37° C. for two hours. For DNAzyme cleavage, the PNA invasion mixture was directly mixed with DNAzymes (or control oligonucleotides) in 10 mM MOPS buffer at pH 7.5 to final concentrations of 5 nM plasmid, 200 nM each of PNAs, 2 μM each of DNAzymes (or control oligonucleotides), 1 mM ZnCl₂, 20 mM MnCl₂, 10 mM MgCl₂, and 37.5 mM NaCl. The mixture was then incubated at room temperature overnight. Prior to post-PANDA procedures, PANDA-treated plasmids were heated to 80° C. for 20 minutes and cooled to room temperature.

Nicking activity of PANDA: Plasmid 1 was untreated (reaction 1); digested with Nb.BbvCI (NEB®) (reaction 2) or Ncol-HF (NEB®) (reaction 3) following recommended procedures provided by NEB®; treated with PANDA-A negative controls as follows: 13PD1-1 only (reaction 4); PNA1 only (reaction 5); PNA2 only (reaction 6); PNA1 and PNA2 (reaction 7); PNA1 and 13PD1-1 (reaction 8); PNA2 and 13PD1-1 (reaction 9); PNA1, PNA2, and T59 (reaction 10); PNA1, PNA2, and 13PD1-un (reaction 11); PNA1, PNA2, and 13PD1-1i (reaction 12); or treated with PANDA-A (PNA1, PNA2, and 13PD1-1). All reactions were combined with 6× Loading Dye (NEB®) and loaded to 1.2% agarose gel for analysis.

Sequence specificity of PANDA: Plasmid 1 was untreated (uncut) or treated with PANDA-A (full match). Plasmid 1 ml (PNA1 region mismatch), plasmid 1m2 (PNA2 region mismatch), or plasmid 1m3 (PNA1+PNA2 region mismatches) was treated with PANDA-A. All reactions were combined with 6× Loading Dye (NEB®) and loaded to 1.2% agarose gel for analysis.

Customizability and DSB activity of PANDA: Plasmid 2 was untreated (reaction 1) or digested with NcoI-HF (NEB®) (reaction 2) following recommended procedures provided by NEB®; treated with PANDA-A (reaction 3) or PANDA-B (reaction 4); or treated with PANDA-A and PANDA-B followed by clean up using a QIAquick® PCR Purification Kit (QIAGEN®) and post-PANDA digestion with NcoI-HF (NEB®) (reaction 5). All reactions were combined with 6× Loading Dye (NEB®) and loaded onto a 1.2% agarose gel for analysis.

Molecular cloning with PANDA: Plasmid 3 was untreated; digested with NcoI-HF (NEB®) following recommended procedures provided by NEB®; or treated with PANDA-A and PANDA-B. All reactions were combined with 6× Loading Dye (NEB®) and loaded to 1.2% agarose gel for analysis. Plasmid 3 treated with PANDA-A and PANDA-B was also loaded to another 1.2% agarose gel for purification. To prepare the vector for molecular cloning, the ˜3.6 kb fragment from the gel was excised and extracted using the QIAEX II® Gel Extraction Kit (QIAGEN®). To prepare the insert for molecular cloning, pS3ins-F and pS3ins-R were annealed in 1×T4 DNA Ligase Reaction Buffer (NEB®). Fifty nanograms of the vector was ligated with the insert at 1:3 molar ratio using T4 DNA ligase (NEB®) following recommended procedures provided by NEB® in a scale of 20 μL. Next, 3 μL of ligation mixture was used to transform 50 μL of NEB® 5-alpha Competent E. coli (High Efficiency). Recombinant plasmids were miniprepped using a QIAprep® Miniprep Kit (QIAGEN®) and sequenced.

### Example 2: bis-PNA PANDA

In this example, bis-PNA openers were used (PNA1, PNA2, PNA3, and PNA4) with modified ssDNA-cleaving DNAzymes (13PD1-1, 13PD1-2). Notably, the 13PD1 catalytic domain exhibits enhanced activity for site-specific hydrolysis of ssDNA. The 13PD1-2 catalytic domain was derived via optimization of the 13PD1 DNAzyme binding domain, providing a DNAzyme with enhanced binding affinity to the area invaded by bis-PNA openers (FIG. 1C).

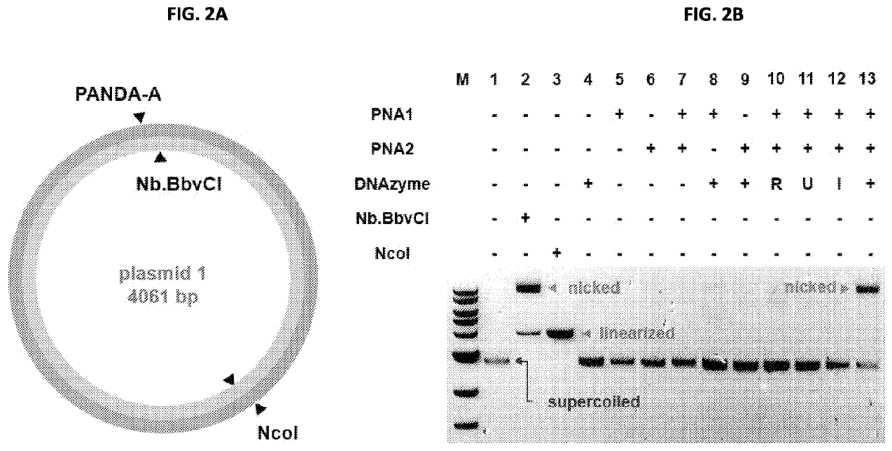

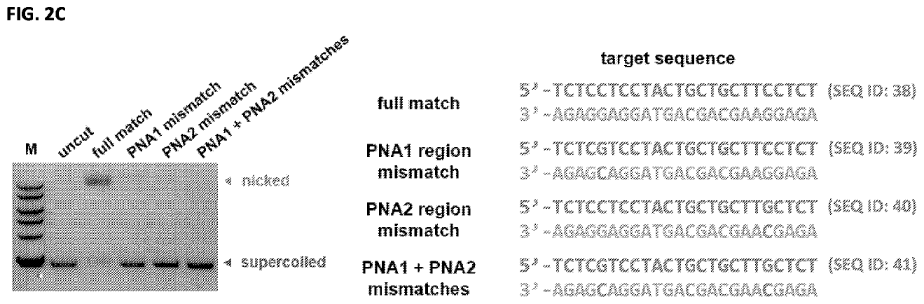

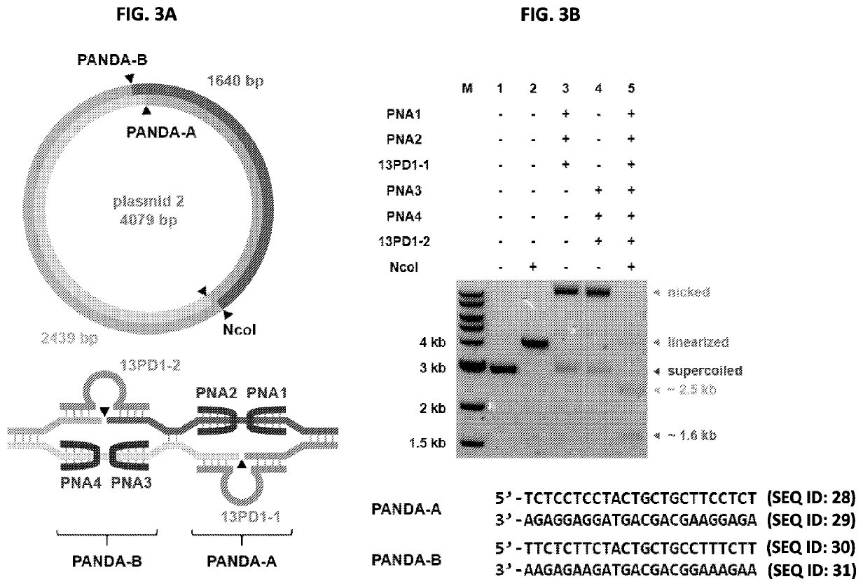

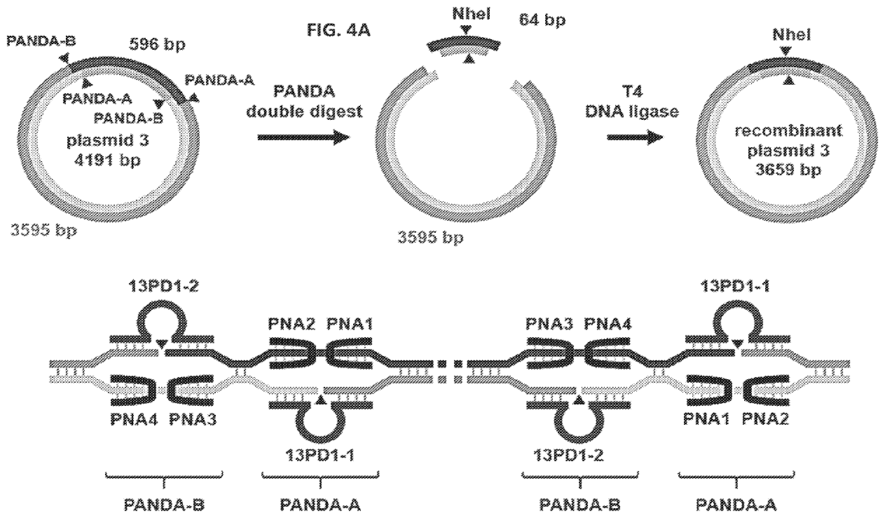

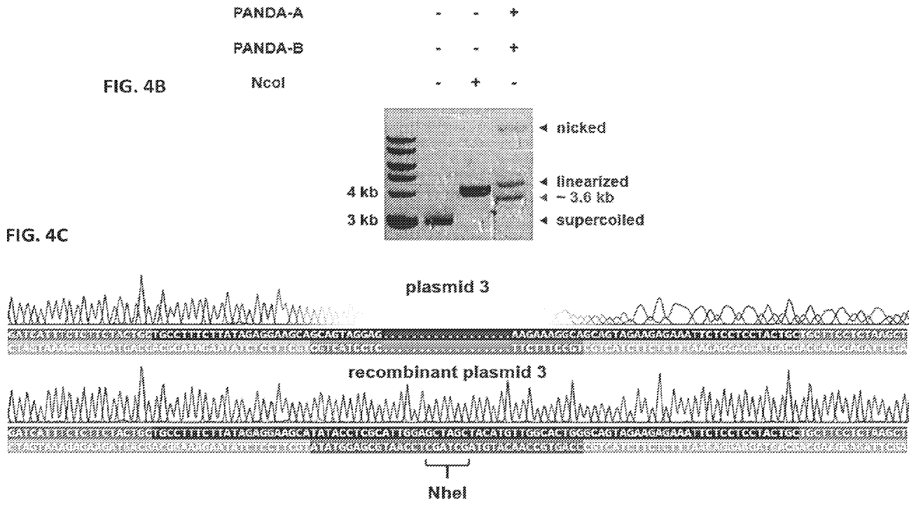

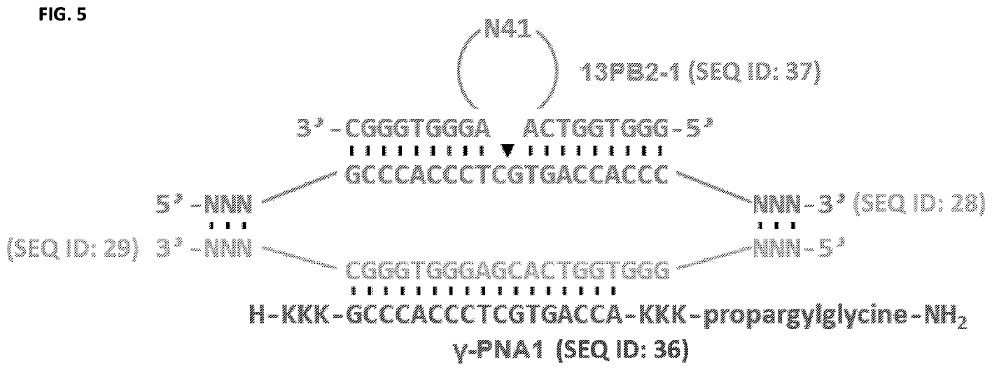

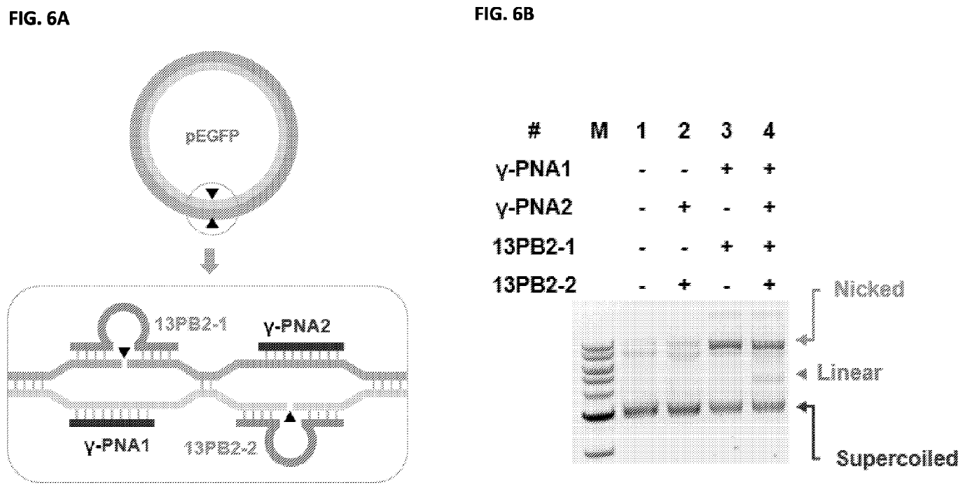

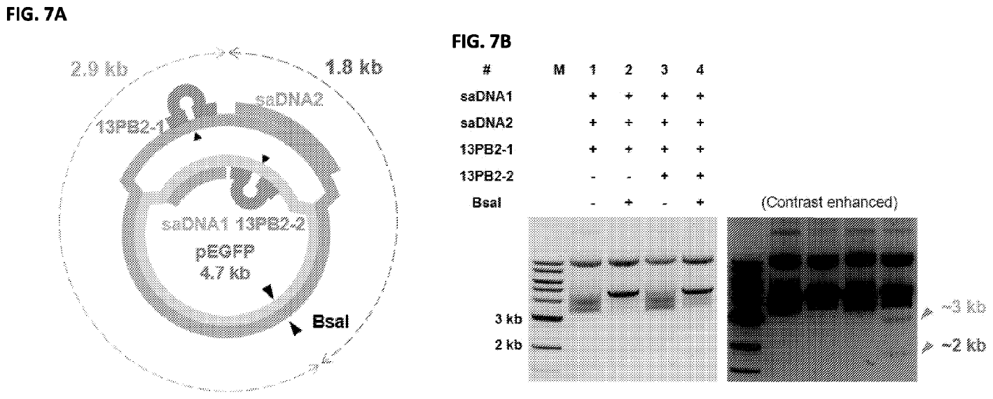

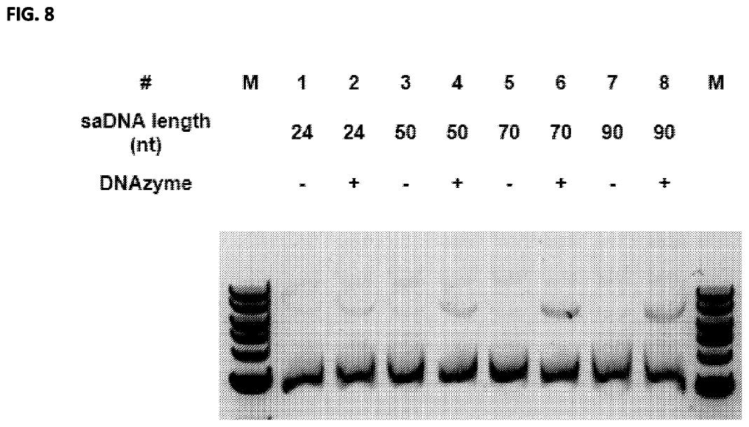

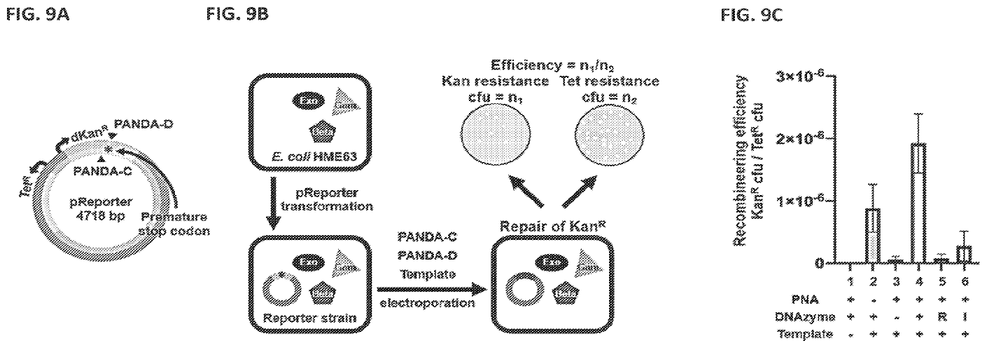

To test the nicking activity of PANDA, plasmids containing example PNA and DNAzyme recognition sequences were generated. First, one PANDA recognition site and a protein nickase recognition site were inserted into a plasmid, ‘plasmid 1’ (FIG. 2A). Because DNAzyme only cleaves one strand of dsDNA, the PANDA system generates a nick on the plasmid. As shown in FIG. 2B, lane 13, plasmid 1 was treated with PNA1 and PNA2 followed by DNAzyme 13PD1-1 (denoted as “+”; PANDA-A treatment; see PANDA treatment in Example 1 for detailed procedures). The band in lane 13 shifted to the same position as the top bands in lane 2 and lane 14, which is the plasmid treated with the protein nickase Nb.BbvCI. This result shows that PANDA generated a nick on the plasmid. PANDA-A-treated plasmid 1 was not linearized, compared with the NcoI restriction enzyme linearized band in lane 3. Further, only when the PANDA-A system was correctly assembled, with two PNAs and a target-specific active 13PD1 DNAzyme present, could the plasmid be nicked (lanes 4-9 compared with lane 13). To confirm that the nick is only created specifically by the active DNAzyme, several DNA oligonucleotides were used as negative controls: T59 (denoted as R), an irrelevant DNA oligonucleotide, which has the same length of the DNAzyme (59 nt), but no catalytic activity or sequence relevance to the target; 13PD1-un (denoted as U), an unspecific DNAzyme, which has an active catalytic domain, but unspecific binding arms (thus, the unspecific DNAzyme may still catalyze a cleavage reaction on other targets); 13PD1-1i (denoted as I), an inactivated DNAzyme, which has specific binding arms that can recognize the target, but a catalytic domain inactivated by systematic transversions (A to T, T to A, C to G, and G to C; thus, the inactive DNAzyme can still bind the target). None of the negative controls generated nicks on the PNA-invaded plasmids (lane 10-13 compared with lane 13). These results indicate that the nick was generated by the DNAzyme in a sequence-specific manner.

To further investigate the sequence specificity of PANDA with the target, bases at the fourth and twentieth nucleotides of the recognition site where PNA1 and PNA2 bind were mutated, respectively (FIG. 2C). Only a little nicked DNA was observed for the plasmid with a mismatch at the PNA1 binding region, which generated a faint band. However, mismatch(es) at the PNA2 region or both PNA1 and PNA2 regions resulted in almost no nicked DNA. These results indicate a significant decrease in PANDA activity even if there is only one mismatch with the target, which exemplifies the enhanced sequence specificity of the PANDA system.

### Example 3—γ-PNA PANDA

As described above, some or all of the PNA monomers may be modified at the γ position in the polyamide backbone, and sequence-generalizable DNAzymes bearing a 13PB2 catalytic domain (referred to herein as “13PB2” or “13PB2-1” and “13PB2-2”) may be used in tandem. Similar to 13PD1-1 and 13PD1-2, the 13PB2 catalytic domain features high activity for site-specific hydrolysis of ssDNA. However, the target site sequence requirements are less stringent for γ-PNA and 13PB2. In some embodiments, γ-PNA binds to a sequence that is complementary to and/or identical to a sequence flanking a 13PB2 target site sequence, thereby making a single-stranded region exposed on dsDNA and thus available for 13PB2 binding. Regarding the target site sequence requirements for the γ-PNA strand, any sequence can be bound by γ-PNA (FIG. 5). Regarding the target sequence requirements of the DNAzyme-binding domain, only a contiguous target site cytosine-guanosine pair (FIG. 5) is required for 13PB2-1 activity. Using this γ-PNA PANDA system, all other residues in the DNAzyme-binding domain may be changed without a significant loss in activity or specificity.

To demonstrate that chemically modified γ-PNAs and 13PB2 can mediate ssDNA and dsDNA cleavage in vitro, a plasmid containing four γ-PNA PANDA sites in two pairs (FIG. 6A) was treated by corresponding γ-PNA PANDA systems. As shown in FIG. 6B, only a γ-PNA PANDA system including γ-PNA1, γ-PNA2, 13PB2-1, and 13PB2-2 generated a DSB break in pEGFP plasmid, characterized by a digestion reaction resulting in a linear fragment (lane 4). In contrast, use of γ-PNA1 and 13PB2-1 alone (lane 3), or γ-PNA2 and 13PB2-2 alone (lane 2), resulted in ssDNA nicking. These results indicate that the γ-PNA PANDA system is capable of generating ssDNA breaks and/or mimicking restriction enzymes to generate DSBs.

The γ-PNA and DNAzyme sequence binding domains used in this example are as follows (with the binding domains underlined):

γ-PNA1:
(SEQ ID NO: 36)
H-KKK-GCCCACCCTCGTGACCA-KKK-propargylglycine-NH₂
13PB2-1:
(SEQ ID NO: 37)
5′GGGTGGTCACTATGACACTTATTATAAATATGCTAGTAACGGATAGGT
TGAGGGTGGGC
γ-PNA2:
(SEQ ID NO: 59)
H-KKK-AGCACTGCACGCCGTAG-KKK-propargylglycine-NH₂
13PB2-2:
(SEQ ID NO: 60)
5′GACCTACGGCTATGACACTTATTATAAATATGCTAGTAACGGATAGGT
TGTGCAGTGCT

### Example 4—DANDA

In another example, PNA is replaced in PANDA with long single-stranded DNA (ssDNA) homologous to the target. The ssDNAs are also referred to herein as “assisting strands” or “saDNAs”. To enable DNAzyme activity on dsDNA, saDNA strand invasion was used to open dsDNA and expose unpaired bases for DNAzymes to bind. Bases exposed by saDNA invasion can be bound by DNAzyme, which can thereafter accept dsDNA as its substrate and cleave the exposed strand, generating a nick on the dsDNA. In this example, since the affinity of saDNA toward dsDNA is lower than that of PNA, heat is used to assist saDNA invasion. Similar to the standard and γ-PNA PANDA systems, DANDA enables two DNAzymes (e.g., two 13PD1-1 DNAzymes) to cleave both strands of the dsDNA (double-nicking) of a target plasmid (e.g., plasmid 1), in addition to site-specific double strand breaks (DSB) (FIG. 7A-7B). In other words, one DNAzyme can be used to cleave one strand of the dsDNA, while the other DNAzyme can be used to cleave the complementary DNA strand, resulting in double-nicking or site-specific DSB. FIG. 7A and FIG. 7B shows this programmable DNA nicking system, using saDNA1 (SEQ ID NO: 42) and saDNA2 (SEQ ID NO: 43) to open the dsDNA and expose unpaired bases for DNAzymes 13PB2-1 (SEQ ID NO: 37) and 13PB2-2 (SEQ ID NO: 60). As shown in FIG. 7B, only pEGFP plasmid incubation with saDNA1, saDNA2, 13PB2-1, and 13PB2-2 (and not saDNA1 & 13PB2-1 alone, or saDNA2 & 13PB2-2 alone) generates ˜3 kb and ˜2 kb dsDNA fragments after BsaI treatment, indicating a DSB break generated by the DANDA system before the BsaI treatment. Thus, the DANDA system is capable of generating ssDNA breaks and/or mimicking restriction enzymes to generate D SBs.

The saDNA activity assay shown in FIG. 7A-7B requires a two-step preparation: a first saDNA invasion step, followed by a second 13PD1-1 DNAzyme cleavage reaction step. Reaction conditions for the first saDNA invasion step include: 10 nM target plasmid, 10 μM each saDNA, 630 mM NaCl, 2.5 mM MgCl2, 10 mM MOPS buffer pH 7.5 (heat to 75° C., then slowly cool to room temperature). Reaction conditions for the second 13PD1-1 DNAzyme cleavage reaction step include: 1 mM ZnCl2, 20 mM MnCl2, 10 mM MgCl2, 10 μM each DNAzyme, 10 mM MOPS buffer pH 7.5 (indicated as final concentration), and incubation at room temperature overnight.

Sequences for saDNA1 and saDNA2 are shown below:

saDNA1:
(SEQ ID NO: 42)
AAGCTGACCCTGAAGTTCATCTGCACCACCGGCAAGCTGCCCGTGCCCTG
GCCCACCCTCGTGACCACCC
saDNA2:
(SEQ ID NO: 43)
GGCGGACTTGAAGAAGTCGTGCTGCTTCATGTGGTCGGGGTAGCGGCTGA
AGCACTGCACGCCGTAGGTC

To demonstrate the potential of DANDA for in vitro DNA nicking and cleavage, various activity assays were conducted. In order to evaluate the effect of the length of the saDNA, cleavage assays were carried out with saDNA strands ranging in length from 24 nucleotides to 90 nucleotides. FIG. 8 shows saDNAs of increasing length including: saDNA3 (SEQ ID NO: 44) (lane 1 & 2), saDNA4 (SEQ ID NO: 45) (lane 3 & 4), saDNA5 (SEQ ID NO:46) (lanes 5 & 6), and saDNA6 (SEQ ID NO: 47) (lane 7 & 8). Increasing length of saDNA was shown to bias the reactant/product equilibrium toward nicked product (FIG. 8). This observation may be attributed to enhanced thermodynamic stability of the DNAzyme/target complex observed for extended base pairing lengths in the 10-70 nucleotide range.

### Example 5—PANDA-Based Recombineering

In this example, PANDA-based recombineering in living bacteria was demonstrated (FIG. 9A-9C). Specifically, PANDA was used to repair a premature stop codon in a Kan resistance gene in E. coli HME63. FIG. 9A shows a schematic for pReporter, which has recognition sites for PANDA-C and PANDA-D on a dead kanamycin gene (containing a premature stop codon) and a functional tetracycline resistance gene as a selection marker for cells bearing the pReporter. FIG. 9B depicts the workflow including generating the reporter strain and PANDA treatment of the strain. The reporter strain includes Exo, Gamma, and Beta, which combine to permit homologous recombination. FIG. 9C shows that recombineering efficiency is calculated from dividing kanamycin-resistance (Kan^{R}) cfu (edited cells) from tetracycline-resistance (Tet^{R}) cfu (total cells with pReporter). Further, FIG. 9C shows that when the incubation step includes PNA, DNAzyme, and a template strand (lane 4), the cells recover Kanamycin resistance. Thus, PANDA-based systems may be used in living bacteria, leveraging endogenous repair pathways such as homologous recombination. This makes PANDA a highly specific tool for sequence recognition and cleavage, for use in both in vitro and in vivo.

Experimental procedures for the PANDA-based recombineering experiment shown in FIG. 9A-9C included plasmid preparation, reporter strain preparation, PANDA electroporation, plating the cells, in addition to various other procedures known in the art. Plasmid preparation was carried out with a NEBuilder HiFi DNA Assembly Cloning Kit (NEB®) following procedures provided by the manufacturer. Briefly, two PCR fragments are amplified from pCRII-TOPO (ThermoFisher®) using primers dKanRins1-F and dKanRins1-R, dKanRins2-F and dKanRins2-R, respectively. The fragments are used as templates for a second-round PCR using primers dKanRins1-F and dKanRins3-R, dKanRins3-F and dKanRins2-R, respectively. For pReporter assembly, the two fragments from the second-round PCR was assembled with pBR322 (NEB®) digested by AatII and PstI (NEB). Notably, E. coli HME63 strain (ATCC®) was used for recombineering experiments.

To prepare the reporter strain, pReporter plasmid was electroporated into electrocompetent HME63 cells following standard transformation procedures. The reporter strain was kept in 25% glycerol at −80° C. until usage. For PANDA-based recombineering, cells of the reporter strain were recovered by inoculating the glycerol stock on a tetracycline-containing plate and incubating overnight (˜24 h) at 30° C. Then, a single colony was obtained from the plate and inoculated to 3 mL LB medium containing both tetracycline and ampicillin. Cells were grown at 30° C. overnight (˜16 h) with shaking (200 rpm). Then, 900 μL of the overnight culture was diluted to 90 mL and grown at the same condition until the A600 reached 0.4-0.5. The culturing temperature was then increased to 42° C. for 15 min for Lambda-red induction. The cells were immediately chilled in an ice-water slurry for 5-10 min, washed twice by sterile water and concentrated to 900 μL.

For electroporation of PANDA-C (PNA1, PNA2, and I-R3-1) and PANDA-D (PNA3, PNA4, and I-R3-2), cells were aliquoted in 50 μL, and total 2 μL mixture of PNA oligos (PNA1˜PNA4, 5 pmole each), DNAzyme oligos (I-R3-1 and I-R3-2, 50 pmole each) or control oligos (100 pmole of oligo-dT28 (indicated as R in FIG. 9C) or 50 pmole each of I-R3-1i and I-R3-2i (indicated as I in FIG. 9C)), and template oligo (Template Kan, 5 pmole) was added to cells. Cells were electroporated using Gene Pulser Xcell Microbial System (Bio-rad®) at 1.8 kV and immediately resuspended in 1 ml of SOC medium. 2.5 μL of 0.2 M ZnCl₂ was then added to the medium for DNAzyme activity. Cells were recovered at 30° C. for 1.5 h with shaking (200 rpm). For each treatment group, cells were plated on two LB agar plates containing kanamycin and tetracycline, respectively, with appropriate dilutions. Plates were incubated at 30° C. overnight (˜24 h) before cfu counting.

In various embodiments, the following sequences are used with the above PANDA-based recombineering system.

Primers:
dKanRins1-F (SEQ ID NO: 48):
5′TGATAATAATGGTTTCTTAGACGTTAGCTTGCAGTGGGCTTACATG
dKanRins1-R (SEQ ID NO: 49):
5′TGTCTCCTCCGTTGAAGCTTCCTCTGTAGCCGGATCAAGCGTATGC
dKanRins2-F (SEQ ID NO: 50):
5′TGTTCTCTTCGTTGAAGCCTTTCTTGCAAGCGAAACATCGCATCGA
dKanRins2-R (SEQ ID NO: 51):
5′GTTGCCATTGCTGCATCAGAAGAACTCGTCAAGAAGGCG
dKanRins3-F (SEQ ID NO: 52):
5’GAGGAGACATGTTCTCTTCGTTGAAGCCTTTCTTG
dKanRins3-R (SEQ ID NO: 53):
5′GAAGAGAACATGTCTCCTCCGTTGAAGCTTCC
I-R3 DNAzymes:
I-R3-1 (SEQ ID NO: 54):
5′CAGAGGAAGTAGTTGAGCTGGAGGAGAC
I-R3-2 (SEQ ID NO: 55):
5′CAAGAAAGGTAGTTGAGCTGAAGAGAAC
Control oligonucleotides:
I-R3-1i (SEQ ID NO: 56):
5′CAGAGGAAGATCAACTCGAGGAGGAGAC
I-R3-2i (SEQ ID NO: 57):
5′CAAGAAAGGATCAACTCGAGAAGAGAAC
oligo-dT28 (SEQ ID NO: 58):
5′TTTTTTTTTTTTTTTTTTTTTTTTTTTT
Homologous recombination template:
Kan Template (SEQ ID NO: 61):
5′GTACGTGCTCGCTCGATGCGATGTTTCGCTTGGTGGTCGAATGGGCAG
GTAGCCGGATCAAGCGTATGCAGCCGCCGCATT

In view of the many possible embodiments to which the principles of the disclosed subject matter may be applied, it should be recognized that the illustrated embodiments are only examples and should not be taken as limiting the scope of the disclosure. Rather, the scope of the disclosure is defined by the following claims.

1. A system for genetic engineering, comprising:
one or more catalytic nucleic acids, comprising:
one or more catalytic domains; and
one or more binding domains, wherein the one or more binding domains comprise a sequence complementary to and/or identical to a sequence flanking a target site and/or within the target site; and
one or more catalytic nucleic acid-assisting reagents, comprising a sequence that specifically binds a sequence that is complementary to and/or identical to a sequence flanking the target site and/or within the target site.

2. The system of claim 1, wherein the one or more catalytic nucleic acids comprise DNAzymes, ribozymes or molecules that comprise a catalytic domain with DNA cleavage, nucleoside excision, phosphorylation, or ligation activity.

3. The system of claim 1, wherein the one or more catalytic nucleic acid-assisting reagents comprise peptide nucleic acids (PNAs), PNA analogs, and/or single-stranded assisting DNAs (saDNAs).

4. The system of claim 2, wherein the catalytic domain of the DNAzyme comprises DNA hydrolysis activity.

5. The system of claim 2, wherein the one or more DNAzymes comprise a 13PD1 catalytic domain sequence, a 13PB2 catalytic domain sequence, or a I-R3 catalytic domain sequence.

6. The system of claim 5, wherein the 13PD1 catalytic domain sequence comprises nucleic acids 10-50 of SEQ ID NO: 5, or wherein the 13PB2 catalytic domain sequence comprises nucleic acids 10-50 of SEQ ID NO: 37, or wherein the I-R3 catalytic domain sequence comprises nucleic acids 10-19 of SEQ ID NO: 54.

7. The system claim 1, wherein a first binding domain specifically binds a sequence flanking the target site, and a second binding domain specifically binds a sequence flanking the target site on the opposite side compared with the first binding domain.

8. (canceled)

9. The system of claim 3, wherein:
(a) the PNA comprises bis-PNA, pseudo-complementary PNA (pc-PNA), tail-clamp PNA (tc-PNA), and/or γ-PNA, and/or
(b) the PNA analog comprises a polyacrylate nucleic acid analog and/or a nucleobase-containing polymer with a polyester, polyvinyl, and/or polyamide backbone.

10. The system of claim 1, wherein:
the one or more catalytic nucleic acid-assisting reagents form a Watson-Crick and/or Hoogsteen base pair with a sequence that is complementary to and/or identical to a sequence flanking the target site and/or within the target site; and/or
the one or more catalytic nucleic acid-assisting reagent sequences form a double helix and/or a triple helix, with a sequence complementary to the sequence flanking a target site and/or the target site sequence.

11. (canceled)

12. A system for genetic engineering, comprising:
one or more DNAzymes, comprising:
a catalytic domain, comprising nucleic acids 10-50 of SEQ ID NO: 5, nucleic acids 10-50 of SEQ ID NO: 37, or nucleic acids 10-19 of SEQ ID NO: 54; and
two binding domains, wherein a first binding domain specifically binds a sequence flanking the target site, and a second binding domain specifically binds a sequence flanking the target site on the opposite side compared with the first binding domain; and
one or more peptide nucleic acids (PNAs), PNA analogs, or dsDNA minor-groove-binding polyamides, comprising a sequence that specifically binds and forms a double helix and/or triple helix, with a sequence that is complementary to a sequence flanking a target site and/or the target site sequence.

13. A kit comprising the system of claim 1.

14. (canceled)

15. A method of genetic engineering, comprising contacting the target nucleic acid with the system of claim 1 under conditions sufficient for specific binding of the one or more catalytic nucleic acid-assisting reagents to the target nucleic acid and under conditions sufficient for catalytic nucleic acid activity.

16. A method of genetic engineering, comprising contacting a target nucleic acid with the system of claim 12 under conditions sufficient for specific binding of the one or more PNAs to the target nucleic acid and under conditions sufficient for DNAzyme activity.

17. A method of genetic engineering, comprising:
contacting a target nucleic acid with one or more catalytic nucleic acid-assisting reagents, wherein:
the one or more catalytic nucleic acid-assisting reagents comprise a sequence that specifically binds a sequence that is complementary to and/or identical to a sequence flanking a target site and/or within a target site; and
the contacting occurs under conditions sufficient for specific binding between the target nucleic acid and the one or more catalytic nucleic acid-assisting reagents, generating a catalytic nucleic acid-assisting reagent-bound target nucleic acid; and
contacting the catalytic nucleic acid-assisting reagent-bound target nucleic acid with one or more catalytic nucleic acids; wherein:
the one or more catalytic nucleic acids comprise:
one or more catalytic domains; and
one or more binding domains, wherein the one or more binding domains comprise a sequence complementary to a sequence flanking the target site; and
the contacting occurs under conditions sufficient for catalytic nucleic acid activity.

18. The method of claim 17, wherein the one or more catalytic nucleic acids comprise DNAzymes and/or wherein the one or more catalytic nucleic acid-assisting reagents comprise peptide nucleic acids (PNAs) or analogs thereof.

19. (canceled)

20. The method of claim 17, wherein:
the conditions sufficient for specific binding between the target nucleic acid and the one or more PNAs comprise a pH of 6-8, low or no salts, and 4° C.-40° C.;
the target nucleic acid is contacted with the one or more PNAs for at least 20 minutes;
the conditions sufficient for DNAzyme activity comprise a pH of 7.5, 1 mM ZnCl₂, 20 mM MnCl₂, and 25° C.; and/or
the PNA-bound target nucleic acid is contacted with the DNAzyme for at least 40 minutes, 1 hour, 2 hours, 4 hours, 6 hours, 8 hours, or overnight.

21-24. (canceled)

25. The method of claim 18, wherein the one or more DNAzymes comprise at least two DNAzymes.

26. The method of claim 18, wherein the one or more DNAzymes and the one or more PNAs comprise a ratio of two PNAs to one DNAzyme.

27. The method of claim 17, wherein the target nucleic acid is a double-stranded DNA.

28-30. (canceled)

31. The method of claim 17, wherein the method is performed in vitro, ex vivo, in vivo, or in cellulo.

32-33. (canceled)

34. The method of claim 17, wherein the one or more catalytic nucleic acid-assisting reagents comprise single-stranded assisting DNA (saDNA), wherein the saDNA generates ssDNA regions on the target nucleic acid to enable catalytic nucleic acid activity.

35. The method of claim 34, wherein:
conditions sufficient for enhancing saDNA invasion of the target comprise a pH of 6-8 and elevated salt concentration;
conditions sufficient for enhancing saDNA invasion of the target comprise incubation at 60-80° C.;
extension of the saDNA to at least 50 nucleotides enhances saDNA invasion of the target; and/or
extension of the saDNA to at least 70 nucleotides enhances saDNA invasion of the target.

36-41. (canceled)


SEQUENCE LISTING (61 sequences)

SEQ ID NO: 1 - DNA, 19 bp, Artificial Sequence
  synthetic oligonucleotide
  misc_feature (1)..(1): N=Thymidine modified with two lysines
  misc_feature (9)..(11): N=8-amino-3,6-dioxaoctanoyl
  misc_feature (12)..(13): N=pseudoisocytosine
  misc_feature (15)..(16): N=pseudoisocytosine
  misc_feature (18)..(18): N=pseudoisocytosine
  misc_feature (19)..(19): N= thymidine modified with a lysine
  misc_feature (19)..(19): N= thymidine modified with lysine residue
nctcctccnnnnntnntnn

SEQ ID NO: 2 - DNA, 19 bp, Artificial Sequence
  synthetic polynucleotide
  misc_feature (1)..(1): N=Thymidine modified with two lysines
  misc_feature (2)..(2): N=pseudoisocytosine
  misc_feature (4)..(5): N=pseudoisocytosine
  misc_feature (8)..(8): N=pseudoisocytosine
  misc_feature (9)..(11): N=8-amino-3,6-dioxaoctanoyl
  misc_feature (19)..(19): N=thymidine modified with lyseine
nntnnttnnnncttcctcn

SEQ ID NO: 3 - DNA, 19 bp, Artificial Sequence
  synthetic polynucleotide
  misc_feature (1)..(1): N-Thymidine modified with two lysines
  misc_feature (3)..(3): N= pseudoisocytosine
  misc_feature (7)..(8): n=pseudoisocytosine
  misc_feature (9)..(11): n=8-amino-3,6-dioxaoctanoyl
  misc_feature (19)..(19): n=thymidine modified with lysine
ntntttnnnnncctttctn

SEQ ID NO: 4 - DNA, 19 bp, Artificial Sequence
  synthetic polynucleotide
  misc_feature (1)..(1): n=Thymidine modified with douyble lyseine
  misc_feature (9)..(11): n=8-amino-3,6-dioxaoctanoyl
  misc_feature (12)..(12): n=pseudoisocytosine
  misc_feature (15)..(15): n=pseudoisocytosine
  misc_feature (17)..(17): n=pseudoisocytosine
  misc_feature (19)..(19): n=Thymidine modified with lysine
ntctcttcnnnnttntntn

SEQ ID NO: 5 - DNA, 59 bp, Artificial Sequence
  synthetic polynucleotide
agaggaagctataccgggcaactattgcctcgtcatcgctattttctgcgaggaggaga

SEQ ID NO: 6 - DNA, 59 bp, Artificial Sequence
  synthetic polynucleotide
aagaaaggctataccgggcaactattgcctcgtcatcgctattttctgcgagaagagaa

SEQ ID NO: 7 - DNA, 59 bp, Artificial Sequence
  synthetic polynucleotide
agaggaagcatatggcccgttgataacggagcagtagcgataaaagacgcaggaggaga

SEQ ID NO: 8 - DNA, 59 bp, Artificial Sequence
  synthetic polynucleotide
catctcttctataccgggcaactattgcctcgtcatcgctattttctgcgatagtgagt

SEQ ID NO: 9 - DNA, 59 bp, Artificial Sequence
  synthetic polynucleotide
ttttttttttttttttttttttttttttttttttttttttttttttttttttttttttt

SEQ ID NO: 10 - DNA, 22 bp, Artificial Sequence
  synthetic polynucleotide
cggcacctcgctaacggattcg

SEQ ID NO: 11 - DNA, 22 bp, Artificial Sequence
  synthetic polynucleotide
caactcggtcgccgcatacacg

SEQ ID NO: 12 - DNA, 36 bp, Artificial Sequence
  synthetic polynucleotide
tgacacacgtagtggtaaacaggtggcaagcgtcgc

SEQ ID NO: 13 - DNA, 36 bp, Artificial Sequence
  synthetic oligonucleotide
agacacactacgtgttgttgcatggtctggtggtgc

SEQ ID NO: 14 - DNA, 90 bp, Artificial Sequence
  synthetic oligonucleotide
cggcacctcgctaacggattcgcctcagccgtctcctcctactgctgcttcctctcggaatgctacttcgtgtatgcggcgaccgagttg

SEQ ID NO: 15 - DNA, 90 bp, Artificial Sequence
  synthetic oligonucleotide
cggcacctcgctaacggattcgcctcagccgtctcgtcctactgctgcttcctctcggaatgctacttcgtgtatgcggcgaccgagttg

SEQ ID NO: 16 - DNA, 90 bp, Artificial Sequence
  synthetic oligonucleotide
cggcacctcgctaacggattcgcctcagccgtctcctcctactgctgcttgctctcggaatgctacttcgtgtatgcggcgaccgagttg

SEQ ID NO: 17 - DNA, 90 bp, Artificial Sequence
  synthetic oligonucleotide
cggcacctcgctaacggattcgcctcagccgtctcgtcctactgctgcttgctctcggaatgctacttcgtgtatgcggcgaccgagttg

SEQ ID NO: 18 - DNA, 108 bp, Artificial Sequence
  synthetic oligonucleotide
cggcacctcgctaacggattcggaattcatctcctcctactgctgcttcctctataagaaaggcagcagtagaagagaaatgatcacgtgtatgcggcgaccgagttg

SEQ ID NO: 19 - DNA, 108 bp, Artificial Sequence
  synthetic oligonucleotide
gtaaacaggtggcaagcgtcgcttattaaaagaaaggcagcagtagaagagaaattctcctcctactgctgcttcctctaagctgtgcaccaccagaccatgcaacaa

SEQ ID NO: 20 - DNA, 90 bp, Artificial Sequence
  synthetic oligonucleotide
tgcctttcttatagaggaagcatatacctcgcattggagctagctacatgttggcactgggcagtagaagagaaattctcctcctactgc

SEQ ID NO: 21 - DNA, 38 bp, Artificial Sequence
  synthetic oligonucleotide
ccagtgccaacatgtagctagctccaatgcgaggtata

SEQ ID NO: 22 - DNA, 90 bp, Artificial Sequence
  synthetic oligonucleotide
caactcggtcgccgcatacacgaagtagcattccgagaggaagcagcagtaggaggagacggctgaggcgaatccgttagcgaggtgccg

SEQ ID NO: 23 - DNA, 90 bp, Artificial Sequence
  synthetic oligonucleotide
caactcggtcgccgcatacacgaagtagcattccgagaggaagcagcagtaggacgagacggctgaggcgaatccgttagcgaggtgccg

SEQ ID NO: 24 - DNA, 90 bp, Artificial Sequence
  synthetic oligonucleotide
caactcggtcgccgcatacacgaagtagcattccgagagcaagcagcagtaggaggagacggctgaggcgaatccgttagcgaggtgccg

SEQ ID NO: 25 - DNA, 90 bp, Artificial Sequence
  synthetic oligonucleotide
caactcggtcgccgcatacacgaagtagcattccgagagcaagcagcagtaggacgagacggctgaggcgaatccgttagcgaggtgccg

SEQ ID NO: 26 - DNA, 108 bp, Artificial Sequence
  synthetic oligonucleotide
caactcggtcgccgcatacacgtgatcatttctcttctactgctgcctttcttatagaggaagcagcagtaggaggagatgaattccgaatccgttagcgaggtgccg

SEQ ID NO: 27 - DNA, 225 bp, Artificial Sequence
  synthetic oligonucleotide
caactcggtcgccgcatacacgtgatcatttctcttctactgctgcctttcttatagaggaagcagcagtaggaggagatgaattccgaatccgttagcgaggtgccggtggtaaacaggtggcaagcgtcgcttattaaaagaaaggcagcagtagaagagaaattctcctcctactgctgcttcctctaagctgtgcaccaccagaccatgcaacaacacgta

SEQ ID NO: 28 - DNA, 24 bp, Artificial Sequence
  synthetic oligonucleotide
tctcctcctactgctgcttcctct

SEQ ID NO: 29 - DNA, 24 bp, Artificial Sequence
  synthetic oligonucleotide
agaggaagcagcagtaggaggaga

SEQ ID NO: 30 - DNA, 24 bp, Artificial Sequence
  synthetic oligonucleotide
ttctcttctactgctgcctttctt

SEQ ID NO: 31 - DNA, 24 bp, Artificial Sequence
  synthetic oligonucleotide
aagagaagatgacgacggaaagaa

SEQ ID NO: 32 - DNA, 107 bp, Artificial Sequence
  synthetic oligonucleotide
gatcatttctcttctactgctgcctttcttatagaggaagcagcagtaggagaagaaaggcagcagtagaagagaaattctcctcctactgctgcttcctctaagct

SEQ ID NO: 33 - DNA, 107 bp, Artificial Sequence
  synthetic oligonucleotide
ctagtaaagagaagatgacgacggaaagaatatctccttcgtcgtcatcctcttctttccgtcgtcatcttctctttaagaggaggatgacgacgaaggagattcga

SEQ ID NO: 34 - DNA, 125 bp, Artificial Sequence
  synthetic oligonucleotide
gatcatttctcttctactgctgcctttcttatagaggaagcatatacctcgcattggagctagctacatgttggcactgggcagtagaagagaaattctcctcctactgctgcttcctctaagct

SEQ ID NO: 35 - DNA, 125 bp, Artificial Sequence
  synthetic oligonucleotide
ctagtaaagagaagatgacgacggaaagaatatctccttcgtatatggagcgtaacctcgatcgatgtacaaccgtgacccgtcatcttctctttaagaggaggatgacgacgaaggagattcga

SEQ ID NO: 36 - DNA, 21 bp, Artificial Sequence
  synthetic oligonucleotide
  misc_feature (1)..(1): n=guanidine modified with three lysines
  misc_feature (18)..(20): n=Lysine
  misc_feature (21)..(21): n=propargylglycine-NH2
ncccaccctcgtgaccannnn

SEQ ID NO: 37 - DNA, 59 bp, Artificial Sequence
  synthetic oligonucleotide
gggtggtcactatgacacttattataaatatgctagtaacggataggttgagggtgggc

SEQ ID NO: 38 - DNA, 24 bp, Artificial Sequence
  synthetic oligonucleotide
tctcctcctactgctgcttcctct

SEQ ID NO: 39 - DNA, 24 bp, Artificial Sequence
  synthetic oligonucleotide
tctcgtcctactgctgcttcctct

SEQ ID NO: 40 - DNA, 24 bp, Artificial Sequence
  synthetic oligonucleotide
tctcctcctactgctgcttgctct

SEQ ID NO: 41 - DNA, 24 bp, Artificial Sequence
  synthetic oligonucleotide
tctcgtcctactgctgcttgctct

SEQ ID NO: 42 - DNA, 70 bp, Artificial Sequence
  synthetic oligonucleotide
aagctgaccctgaagttcatctgcaccaccggcaagctgcccgtgccctggcccaccctcgtgaccaccc

SEQ ID NO: 43 - DNA, 70 bp, Artificial Sequence
  synthetic oligonucleotide
ggcggacttgaagaagtcgtgctgcttcatgtggtcggggtagcggctgaagcactgcacgccgtaggtc

SEQ ID NO: 44 - DNA, 24 bp, Artificial Sequence
  synthetic oligonucleotide
tctcctcctactgctgcttcctct

SEQ ID NO: 45 - DNA, 50 bp, Artificial Sequence
  synthetic oligonucleotide
cgcctcagccgtctcctcctactgctgcttcctctcggaatgctacttcg

SEQ ID NO: 46 - DNA, 70 bp, Artificial Sequence
  synthetic oligonucleotide
ctaacggattcgcctcagccgtctcctcctactgctgcttcctctcggaatgctacttcgtgtatgcggc

SEQ ID NO: 47 - DNA, 90 bp, Artificial Sequence; identical to SEQ ID NO: 14
  synthetic oligonucleotide

SEQ ID NO: 48 - DNA, 46 bp, Artificial Sequence
  synthetic oligonucleotide
tgataataatggtttcttagacgttagcttgcagtgggcttacatg

SEQ ID NO: 49 - DNA, 46 bp, Artificial Sequence
  synthetic oligonucleotide
tgtctcctccgttgaagcttcctctgtagccggatcaagcgtatgc

SEQ ID NO: 50 - DNA, 46 bp, Artificial Sequence
  synthetic oligonucleotide
tgttctcttcgttgaagcctttcttgcaagcgaaacatcgcatcga

SEQ ID NO: 51 - DNA, 39 bp, Artificial Sequence
  synthetic oligonucleotide
gttgccattgctgcatcagaagaactcgtcaagaaggcg

SEQ ID NO: 52 - DNA, 35 bp, Artificial Sequence
  synthetic oligonucleotide
gaggagacatgttctcttcgttgaagcctttcttg

SEQ ID NO: 53 - DNA, 32 bp, Artificial Sequence
  synthetic oligonucleotide
gaagagaacatgtctcctccgttgaagcttcc

SEQ ID NO: 54 - DNA, 28 bp, Artificial Sequence
  synthetic oligonucleotide
cagaggaagtagttgagctggaggagac

SEQ ID NO: 55 - DNA, 28 bp, Artificial Sequence
  synthetic oligonucleotide
caagaaaggtagttgagctgaagagaac

SEQ ID NO: 56 - DNA, 28 bp, Artificial Sequence
  synthetic oligonucleotide
cagaggaagatcaactcgaggaggagac

SEQ ID NO: 57 - DNA, 28 bp, Artificial Sequence
  synthetic oligonucleotide
caagaaaggatcaactcgagaagagaac

SEQ ID NO: 58 - DNA, 28 bp, Artificial Sequence
  synthetic oligonucleotide
tttttttttttttttttttttttttttt

SEQ ID NO: 59 - DNA, 17 bp, Artificial Sequence
  synthetic oligonucleotide
  misc_feature (1)..(1): n=Adenine modified with three lysines
  misc_feature (17)..(19): n=lyseine
  misc_feature (20)..(20): n=propargylglycine-NH2
ngcactgcacgccgtan

SEQ ID NO: 60 - DNA, 59 bp, Artificial Sequence
  synthetic oligonucleotide
gacctacggctatgacacttattataaatatgctagtaacggataggttgtgcagtgct

SEQ ID NO: 61 - DNA, 81 bp, Artificial Sequence
  synthetic oligonucleotide
gtacgtgctcgctcgatgcgatgtttcgcttggtggtcgaatgggcaggtagccggatcaagcgtatgcagccgccgcatt